SEQUENCE LISTING (16 sequences)

SEQ ID NO: 1 - DNA, 393 bp, synthetic construct
  source 1..393: mol_type = other DNA
atggctgctcctccagaacccggcgagcctgaggaacggaaggccagctgcaccagcctgcacctgagctactggaagtccctgaagctgctgggctttctggatgtggaaaacaccccttgcgcccggcacagcatcctgtatggcagcctgggaagcgtggtggccggcttcggccatttcctgttcacaagcagaattagaaggtcctgcgacgtgggcgtgggaggattcatcctggtcacactgggctgctggttccactgtagatacaactacgccaagcagagaatccaggagcggatcgccagagaggaaatcaagaaaaaaatcctgtacgagggcacccacctcgaccccgagagaaagcacaacggctcttctagcaattga

SEQ ID NO: 2 - DNA, 357 bp, synthetic construct
  source 1..357: mol_type = other DNA
atggccgctccccccgagccaggcgagcctgaggaaagaaagtccctgaagctgctgggcttcctggacgtggaaaacaccccttgcgccagacacagcatcctgtatggcagcctgggatctgtggtcgccggcttcggccacttcctgttcaccagcagaatccggagaagctgcgacgtgggcgtgggaggctttatcctggtgacactgggatgttggttccactgcagatacaactacgccaagcagcggattcaggagagaatcgcccgcgaggaaatcaagaaaaagatcctctacgagggcacccacctggatcctgaacggaaacataatggctctagcagcaactga

SEQ ID NO: 3 - protein, 130 aa, Homo sapiens
MAAPPEPGEPEERKASCTSLHLSYWKSLKLLGFLDVENTPCARHSILYGSLGSVVAGFGHFLFTSRIRRSCDVGVGGFILVTLGCWFHCRYNYAKQRIQERIAREEIKKKILYEGTHLDPERKHNGSSSN

SEQ ID NO: 4 - protein, 118 aa, Homo sapiens
MAAPPEPGEPEERKSLKLLGFLDVENTPCARHSILYGSLGSVVAGFGHFLFTSRIRRSCDVGVGGFILVTLGCWFHCRYNYAKQRIQERIAREEIKKKILYEGTHLDPERKHNGSSSN

SEQ ID NO: 5 - DNA, 1581 bp, synthetic construct
  source 1..1581: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgagaattcgccaccatggctgctcctccagaacccggcgagcctgaggaacggaaggccagctgcaccagcctgcacctgagctactggaagtccctgaagctgctgggctttctggatgtggaaaacaccccttgcgcccggcacagcatcctgtatggcagcctgggaagcgtggtggccggcttcggccatttcctgttcacaagcagaattagaaggtcctgcgacgtgggcgtgggaggattcatcctggtcacactgggctgctggttccactgtagatacaactacgccaagcagagaatccaggagcggatcgccagagaggaaatcaagaaaaaaatcctgtacgagggcacccacctcgaccccgagagaaagcacaacggctcttctagcaattgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 6 - DNA, 1545 bp, synthetic construct
  source 1..1545: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgagaattcgccaccatggccgctccccccgagccaggcgagcctgaggaaagaaagtccctgaagctgctgggcttcctggacgtggaaaacaccccttgcgccagacacagcatcctgtatggcagcctgggatctgtggtcgccggcttcggccacttcctgttcaccagcagaatccggagaagctgcgacgtgggcgtgggaggctttatcctggtgacactgggatgttggttccactgcagatacaactacgccaagcagcggattcaggagagaatcgcccgcgaggaaatcaagaaaaagatcctctacgagggcacccacctggatcctgaacggaaacataatggctctagcagcaactgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 7 - DNA, 4402 bp, synthetic construct
  source 1..4402: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgagaattcgccaccatggctgctcctccagaacccggcgagcctgaggaacggaaggccagctgcaccagcctgcacctgagctactggaagtccctgaagctgctgggctttctggatgtggaaaacaccccttgcgcccggcacagcatcctgtatggcagcctgggaagcgtggtggccggcttcggccatttcctgttcacaagcagaattagaaggtcctgcgacgtgggcgtgggaggattcatcctggtcacactgggctgctggttccactgtagatacaactacgccaagcagagaatccaggagcggatcgccagagaggaaatcaagaaaaaaatcctgtacgagggcacccacctcgaccccgagagaaagcacaacggctcttctagcaattgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 8 - DNA, 4366 bp, synthetic construct
  source 1..4366: mol_type = other DNA
ctgcgcgctcgctcgctcactgaggccgcccgggcaaagcccgggcgtcgggcgacctttggtcgcccggcctcagtgagcgagcgagcgcgcagagagggagtgtagccatgctctaggaagatcaattcggtacaattcacgcgtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctctagcctcgagaattcgccaccatggccgctccccccgagccaggcgagcctgaggaaagaaagtccctgaagctgctgggcttcctggacgtggaaaacaccccttgcgccagacacagcatcctgtatggcagcctgggatctgtggtcgccggcttcggccacttcctgttcaccagcagaatccggagaagctgcgacgtgggcgtgggaggctttatcctggtgacactgggatgttggttccactgcagatacaactacgccaagcagcggattcaggagagaatcgcccgcgaggaaatcaagaaaaagatcctctacgagggcacccacctggatcctgaacggaaacataatggctctagcagcaactgaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggcctaggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 9 - DNA, 2821 bp, synthetic construct
  source 1..2821: mol_type = other DNA
gcctcgtgtaataatatttaattaagaaaaagccgggtgccatagctcatgtctgtaatcccagcactttgggagggtgagacaggaggatcactttagtccaggagtttgagaccagcctgggcaacaaagtgacacaccatcccccacctcccgtctctacaaataattgtttttaagaaggataaaagagtttgaaaagagagacatcaaactgttagttatatttaccttaaaaaggagagaaagaactgagatttttactttcctttctataactgttaaacaattaaaaatggaaatattatatgcaattttgtgaaaataaaaattttaaaataggccaggcgcagtggctcacgcctgtaatcccagcactctggaaaccagaggcaggcgtatcacctgaggtcaggagtttgaggccagcctggccaacatagtgaaaccctttctctactaaaaatacaaaaattagccgggtgtggtggcaggcgcctgtaatcccacaatgtctactctagcaaattttcaaagtcataataaaaataaaataggatgactcggtggtgttttagctggtacttttccagttttccttttacaacataatcaacaatgacttcaaaaggatttccccctccttcatggaatattactaccttcaacacggcctttcagatccggagttcaaagcatagttagcattataatttcagcagagaatccagaattggcaggatatcaccagtagggtgactttttttcttttccctagcaaacagtagagcagtatgtagtttctccccccgcccagacggagtttcgctctttctcccaggctggagtacagtaaaaaacctcaaaaaaaaaaatgtgacacataaatacacaaccttaactgtgtattttactgggattgtagaaaggtatcaaatgggcatgcaagtaaggtccttacggagtaagaaaacgcccaatgaacagtacttaatatgaattcgacatcgttaacactgagcgcttgctgtgtgccagcgcgcagcataattcgaagcagcatgactttgcacaatgacatattctacaagtccagtttcctaaggagtgcccttaggttacatataacctaaacgtgaaatgtagaagtacataaaggaggcataaagttaaaggggaaaaattataaaaattgcattgtattattttgatggctgaaaaatgggctcgaccctgcatgcacgcgttctccattgagctcgacaatatgcagtattttgtttcacgttatgtcgcagtcttagggaggagtgaaacgtggaacacgacttcacatcaaaaatgcacgttgcagaggcatttttcctgttaaagccacttagtaatgaaactataaaagggaggaagtggggtgggcgtgggagccccgtttgcgcaaggcaggcgcgggggactaaatggctgctggtgcagagtggggctgccccggcaccaagggctcgtcccgtggccggaaggacaaaggacacttaggggacgagggccgccgagccagcgcggagcgccttccgggtcggtgggctggggcgggctgggggctggggccgggccgacccccgcggtggggcccaccccgcaggacaaagcagcggccccgcccctcggcgcgcgcccgaggctcgtgcgtgtgagcacgcgcgccggagacggttagaaaagaaagtgtgacccgttcgacggaacaaaggacatgacagcccggccccgtgcggccactgcggagcgttaggaacattccctaaaatggcggccggcgcgtcggaacagggcgggaggcggcggcgcgtgggggcgggacggggaggggcgcggccagccgggcttctgcttccgcgaccccggcggtgcagggcgggtggagtcgcggagtagtccggcggggcgcgcgccgcgggtgggcggtgggtaaggacgttctccgcggagctcctaggcccggagcacgtgtcactggcttctctgccacccatgggcttgaaactattttaactgttcctagtccttatcccaagcctccaacattttaatctaagtggccgggacaaaattgaccaaaactagggggaacataggagtaattgctgagggatgacttgattgtactacgatgagatgaagggttaagatttcttccccatcaccagtagcctccctagctggtgtccctgtctccatactctcctgaatccaccttccactattgtccgctggccctttccagcctgtaaactggacagttttcattcctctgcctaacatccagtcgtcctaatatatacttaagatattctcccagcttggaaattctgctcacttcttgctcttgcttgcttctctggtgggcttccatccaaatttggccatgacatcagatcatatggttaatattaacaaatattgtataaattgttttcatacttaatatccaacaactagactgatcaagacaggaaaaatccctttaaggagctgacattctactggaggaatcaataagcaagacttctactgagccagtcatgcaaagatccaggagaaaggcagcatgtgttcgttcggtaataaccatctcggcttgttagaatggcaaggccactgtacctggactagtggacaagagtaggatatgaaagagcatgggatcatgtagggcctttggagcaatggtaaggattctgagcataatcggaggctacagaggacttattatagtcatctgatacacatttttatgagtagaatcaagatctgaaaggtactg

SEQ ID NO: 10 - DNA, 1818 bp, synthetic construct
  source 1..1818: mol_type = other DNA
caatgtctactctagcaaattttcaaagtcataataaaaataaaataggatgactcggtggtgttttagctggtacttttccagttttccttttacaacataatcaacaatgacttcaaaaggatttccccctccttcatggaatattactaccttcaacacggcctttcagatccggagttcaaagcatagttagcattataatttcagcagagaatccagaattggcaggatatcaccagtagggtgactttttttcttttccctagcaaacagtagagcagtatgtagtttctccccccgcccagacggagtttcgctctttctcccaggctggagtacagtaaaaaacctcaaaaaaaaaaatgtgacacataaatacacaaccttaactgtgtattttactgggattgtagaaaggtatcaaatgggcatgcaagtaaggtccttacggagtaagaaaacgcccaatgaacagtacttaatatgaattcgacatcgttaacactgagcgcttgctgtgtgccagcgcgcagcataattcgaagcagcatgactttgcacaatgacatattctacaagtccagtttcctaaggagtgcccttaggttacatataacctaaacgtgaaatgtagaagtacataaaggaggcataaagttaaaggggaaaaattataaaaattgcattgtattattttgatggctgaaaaatgggctcgaccctgcatgcacgcgttctccattgagctcgacaatatgcagtattttgtttcacgttatgtcgcagtcttagggaggagtgaaacgtggaacacgacttcacatcaaaaatgcacgttgcagaggcatttttcctgttaaagccacttagtaatgaaactataaaagggaggaagtggggtgggcgtgggagccccgtttgcgcaaggcaggcgcgggggactaaatggctgctggtgcagagtggggctgccccggcaccaagggctcgtcccgtggccggaaggacaaaggacacttaggggacgagggccgccgagccagcgcggagcgccttccgggtcggtgggctggggcgggctgggggctggggccgggccgacccccgcggtggggcccaccccgcaggacaaagcagcggccccgcccctcggcgcgcgcccgaggctcgtgcgtgtgagcacgcgcgccggagacggttagaaaagaaagtgtgacccgttcgacggaacaaaggacatgacagcccggccccgtgcggccactgcggagcgttaggaacattccctaaaatggcggccggcgcgtcggaacagggcgggaggcggcggcgcgtgggggcgggacggggaggggcgcggccagccgggcttctgcttccgcgaccccggcggtgcagggcgggtggagtcgcggagtagtccggcggggcgcgcgccgcgggtgggcggtgggtaaggacgttctccgcggagctcctaggcccggagcacgtgtcactggcttctctgccacccatgggcttgaaactattttaactgttcctagtccttatcccaagcctccaacattttaatctaagtggccgggacaaaattgaccaaaactagggggaacataggagtaattgctgagggatgacttgattgtactacgatgagatgaagggttaagatttcttccccatcaccagtagcctccctagctggtgtccctgtctccatactctcctgaatccaccttccactattgtccgctggccctttccagcctgtaaactggacagttttcattcctctgcctaacatccagtcgt

SEQ ID NO: 11 - DNA, 350 bp, Homo sapiens
  source 1..350: mol_type = genomic DNA
tgttaaagccacttagtaatgaaactataaaagggaggaagtggggtgggcgtgggagccccgtttgcgcaaggcaggcgcgggggactaaatggctgctggtgcagagtggggctgccccggcaccaagggctcgtcccgtggccggaaggacaaaggacacttaggggacgagggccgccgagccagcgcggagcgccttccgggtcggtgggctggggcgggctgggggctggggccgggccgacccccgcggtggggcccaccccgcaggacaaagcagcggccccgcccctcggcgcgcgcccgaggctcgtgcgtgtgagcacgcgcgccggagacggttagaa

SEQ ID NO: 12 - DNA, 219 bp, Homo sapiens
  source 1..219: mol_type = genomic DNA
aagaaagtgtgacccgttcgacggaacaaaggacatgacagcccggccccgtgcggccactgcggagcgttaggaacattccctaaaatggcggccggcgcgtcggaacagggcgggaggcggcggcgcgtgggggcgggacggggaggggcgcggccagccgggcttctgcttccgcgaccccggcggtgcagggcgggtggagtcgcggagtagtcc

SEQ ID NO: 13 - DNA, 6733 bp, synthetic construct
  source 1..6733: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagctagaccggtgcctcgtgtaataatatttaattaagaaaaagccgggtgccatagctcatgtctgtaatcccagcactttgggagggtgagacaggaggatcactttagtccaggagtttgagaccagcctgggcaacaaagtgacacaccatcccccacctcccgtctctacaaataattgtttttaagaaggataaaagagtttgaaaagagagacatcaaactgttagttatatttaccttaaaaaggagagaaagaactgagatttttactttcctttctataactgttaaacaattaaaaatggaaatattatatgcaattttgtgaaaataaaaattttaaaataggccaggcgcagtggctcacgcctgtaatcccagcactctggaaaccagaggcaggcgtatcacctgaggtcaggagtttgaggccagcctggccaacatagtgaaaccctttctctactaaaaatacaaaaattagccgggtgtggtggcaggcgcctgtaatcccacaatgtctactctagcaaattttcaaagtcataataaaaataaaataggatgactcggtggtgttttagctggtacttttccagttttccttttacaacataatcaacaatgacttcaaaaggatttccccctccttcatggaatattactaccttcaacacggcctttcagatccggagttcaaagcatagttagcattataatttcagcagagaatccagaattggcaggatatcaccagtagggtgactttttttcttttccctagcaaacagtagagcagtatgtagtttctccccccgcccagacggagtttcgctctttctcccaggctggagtacagtaaaaaacctcaaaaaaaaaaatgtgacacataaatacacaaccttaactgtgtattttactgggattgtagaaaggtatcaaatgggcatgcaagtaaggtccttacggagtaagaaaacgcccaatgaacagtacttaatatgaattcgacatcgttaacactgagcgcttgctgtgtgccagcgcgcagcataattcgaagcagcatgactttgcacaatgacatattctacaagtccagtttcctaaggagtgcccttaggttacatataacctaaacgtgaaatgtagaagtacataaaggaggcataaagttaaaggggaaaaattataaaaattgcattgtattattttgatggctgaaaaatgggctcgaccctgcatgcacgcgttctccattgagctcgacaatatgcagtattttgtttcacgttatgtcgcagtcttagggaggagtgaaacgtggaacacgacttcacatcaaaaatgcacgttgcagaggcatttttcctgttaaagccacttagtaatgaaactataaaagggaggaagtggggtgggcgtgggagccccgtttgcgcaaggcaggcgcgggggactaaatggctgctggtgcagagtggggctgccccggcaccaagggctcgtcccgtggccggaaggacaaaggacacttaggggacgagggccgccgagccagcgcggagcgccttccgggtcggtgggctggggcgggctgggggctggggccgggccgacccccgcggtggggcccaccccgcaggacaaagcagcggccccgcccctcggcgcgcgcccgaggctcgtgcgtgtgagcacgcgcgccggagacggttagaaaagaaagtgtgacccgttcgacggaacaaaggacatgacagcccggccccgtgcggccactgcggagcgttaggaacattccctaaaatggcggccggcgcgtcggaacagggcgggaggcggcggcgcgtgggggcgggacggggaggggcgcggccagccgggcttctgcttccgcgaccccggcggtgcagggcgggtggagtcgcggagtagtccggcggggcgcgcgccgcgggtgggcggtgggtaaggacgttctccgcggagctcctaggcccggagcacgtgtcactggcttctctgccacccatgggcttgaaactattttaactgttcctagtccttatcccaagcctccaacattttaatctaagtggccgggacaaaattgaccaaaactagggggaacataggagtaattgctgagggatgacttgattgtactacgatgagatgaagggttaagatttcttccccatcaccagtagcctccctagctggtgtccctgtctccatactctcctgaatccaccttccactattgtccgctggccctttccagcctgtaaactggacagttttcattcctctgcctaacatccagtcgtcctaatatatacttaagatattctcccagcttggaaattctgctcacttcttgctcttgcttgcttctctggtgggcttccatccaaatttggccatgacatcagatcatatggttaatattaacaaatattgtataaattgttttcatacttaatatccaacaactagactgatcaagacaggaaaaatccctttaaggagctgacattctactggaggaatcaataagcaagacttctactgagccagtcatgcaaagatccaggagaaaggcagcatgtgttcgttcggtaataaccatctcggcttgttagaatggcaaggccactgtacctggactagtggacaagagtaggatatgaaagagcatgggatcatgtagggcctttggagcaatggtaaggattctgagcataatcggaggctacagaggacttattatagtcatctgatacacatttttatgagtagaatcaagatctgaaaggtactggtcgaaattcgccaccatggctgctcctccagaacccggcgagcctgaggaacggaaggccagctgcaccagcctgcacctgagctactggaagtccctgaagctgctgggctttctggatgtggaaaacaccccttgcgcccggcacagcatcctgtatggcagcctgggaagcgtggtggccggcttcggccatttcctgttcacaagcagaattagaaggtcctgcgacgtgggcgtgggaggattcatcctggtcacactgggctgctggttccactgtagatacaactacgccaagcagagaatccaggagcggatcgccagagaggaaatcaagaaaaaaatcctgtacgagggcacccacctcgaccccgagagaaagcacaacggctcttctagcaattgaggtaccacggggtgaactacgcctgaggatcatcgatagatctctagtttaaacgggcccctcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 14 - DNA, 6697 bp, synthetic construct
  source 1..6697: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagctagaccggtgcctcgtgtaataatatttaattaagaaaaagccgggtgccatagctcatgtctgtaatcccagcactttgggagggtgagacaggaggatcactttagtccaggagtttgagaccagcctgggcaacaaagtgacacaccatcccccacctcccgtctctacaaataattgtttttaagaaggataaaagagtttgaaaagagagacatcaaactgttagttatatttaccttaaaaaggagagaaagaactgagatttttactttcctttctataactgttaaacaattaaaaatggaaatattatatgcaattttgtgaaaataaaaattttaaaataggccaggcgcagtggctcacgcctgtaatcccagcactctggaaaccagaggcaggcgtatcacctgaggtcaggagtttgaggccagcctggccaacatagtgaaaccctttctctactaaaaatacaaaaattagccgggtgtggtggcaggcgcctgtaatcccacaatgtctactctagcaaattttcaaagtcataataaaaataaaataggatgactcggtggtgttttagctggtacttttccagttttccttttacaacataatcaacaatgacttcaaaaggatttccccctccttcatggaatattactaccttcaacacggcctttcagatccggagttcaaagcatagttagcattataatttcagcagagaatccagaattggcaggatatcaccagtagggtgactttttttcttttccctagcaaacagtagagcagtatgtagtttctccccccgcccagacggagtttcgctctttctcccaggctggagtacagtaaaaaacctcaaaaaaaaaaatgtgacacataaatacacaaccttaactgtgtattttactgggattgtagaaaggtatcaaatgggcatgcaagtaaggtccttacggagtaagaaaacgcccaatgaacagtacttaatatgaattcgacatcgttaacactgagcgcttgctgtgtgccagcgcgcagcataattcgaagcagcatgactttgcacaatgacatattctacaagtccagtttcctaaggagtgcccttaggttacatataacctaaacgtgaaatgtagaagtacataaaggaggcataaagttaaaggggaaaaattataaaaattgcattgtattattttgatggctgaaaaatgggctcgaccctgcatgcacgcgttctccattgagctcgacaatatgcagtattttgtttcacgttatgtcgcagtcttagggaggagtgaaacgtggaacacgacttcacatcaaaaatgcacgttgcagaggcatttttcctgttaaagccacttagtaatgaaactataaaagggaggaagtggggtgggcgtgggagccccgtttgcgcaaggcaggcgcgggggactaaatggctgctggtgcagagtggggctgccccggcaccaagggctcgtcccgtggccggaaggacaaaggacacttaggggacgagggccgccgagccagcgcggagcgccttccgggtcggtgggctggggcgggctgggggctggggccgggccgacccccgcggtggggcccaccccgcaggacaaagcagcggccccgcccctcggcgcgcgcccgaggctcgtgcgtgtgagcacgcgcgccggagacggttagaaaagaaagtgtgacccgttcgacggaacaaaggacatgacagcccggccccgtgcggccactgcggagcgttaggaacattccctaaaatggcggccggcgcgtcggaacagggcgggaggcggcggcgcgtgggggcgggacggggaggggcgcggccagccgggcttctgcttccgcgaccccggcggtgcagggcgggtggagtcgcggagtagtccggcggggcgcgcgccgcgggtgggcggtgggtaaggacgttctccgcggagctcctaggcccggagcacgtgtcactggcttctctgccacccatgggcttgaaactattttaactgttcctagtccttatcccaagcctccaacattttaatctaagtggccgggacaaaattgaccaaaactagggggaacataggagtaattgctgagggatgacttgattgtactacgatgagatgaagggttaagatttcttccccatcaccagtagcctccctagctggtgtccctgtctccatactctcctgaatccaccttccactattgtccgctggccctttccagcctgtaaactggacagttttcattcctctgcctaacatccagtcgtcctaatatatacttaagatattctcccagcttggaaattctgctcacttcttgctcttgcttgcttctctggtgggcttccatccaaatttggccatgacatcagatcatatggttaatattaacaaatattgtataaattgttttcatacttaatatccaacaactagactgatcaagacaggaaaaatccctttaaggagctgacattctactggaggaatcaataagcaagacttctactgagccagtcatgcaaagatccaggagaaaggcagcatgtgttcgttcggtaataaccatctcggcttgttagaatggcaaggccactgtacctggactagtggacaagagtaggatatgaaagagcatgggatcatgtagggcctttggagcaatggtaaggattctgagcataatcggaggctacagaggacttattatagtcatctgatacacatttttatgagtagaatcaagatctgaaaggtactggtcgaaattcgccaccatggccgctccccccgagccaggcgagcctgaggaaagaaagtccctgaagctgctgggcttcctggacgtggaaaacaccccttgcgccagacacagcatcctgtatggcagcctgggatctgtggtcgccggcttcggccacttcctgttcaccagcagaatccggagaagctgcgacgtgggcgtgggaggctttatcctggtgacactgggatgttggttccactgcagatacaactacgccaagcagcggattcaggagagaatcgcccgcgaggaaatcaagaaaaagatcctctacgagggcacccacctggatcctgaacggaaacataatggctctagcagcaactgaggtaccacggggtgaactacgcctgaggatcatcgatagatctctagtttaaacgggcccctcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattagg

SEQ ID NO: 15 - DNA, 5730 bp, synthetic construct
  source 1..5730: mol_type = other DNA
aactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagctagaccggtcaatgtctactctagcaaattttcaaagtcataataaaaataaaataggatgactcggtggtgttttagctggtacttttccagttttccttttacaacataatcaacaatgacttcaaaaggatttccccctccttcatggaatattactaccttcaacacggcctttcagatccggagttcaaagcatagttagcattataatttcagcagagaatccagaattggcaggatatcaccagtagggtgactttttttcttttccctagcaaacagtagagcagtatgtagtttctccccccgcccagacggagtttcgctctttctcccaggctggagtacagtaaaaaacctcaaaaaaaaaaatgtgacacataaatacacaaccttaactgtgtattttactgggattgtagaaaggtatcaaatgggcatgcaagtaaggtccttacggagtaagaaaacgcccaatgaacagtacttaatatgaattcgacatcgttaacactgagcgcttgctgtgtgccagcgcgcagcataattcgaagcagcatgactttgcacaatgacatattctacaagtccagtttcctaaggagtgcccttaggttacatataacctaaacgtgaaatgtagaagtacataaaggaggcataaagttaaaggggaaaaattataaaaattgcattgtattattttgatggctgaaaaatgggctcgaccctgcatgcacgcgttctccattgagctcgacaatatgcagtattttgtttcacgttatgtcgcagtcttagggaggagtgaaacgtggaacacgacttcacatcaaaaatgcacgttgcagaggcatttttcctgttaaagccacttagtaatgaaactataaaagggaggaagtggggtgggcgtgggagccccgtttgcgcaaggcaggcgcgggggactaaatggctgctggtgcagagtggggctgccccggcaccaagggctcgtcccgtggccggaaggacaaaggacacttaggggacgagggccgccgagccagcgcggagcgccttccgggtcggtgggctggggcgggctgggggctggggccgggccgacccccgcggtggggcccaccccgcaggacaaagcagcggccccgcccctcggcgcgcgcccgaggctcgtgcgtgtgagcacgcgcgccggagacggttagaaaagaaagtgtgacccgttcgacggaacaaaggacatgacagcccggccccgtgcggccactgcggagcgttaggaacattccctaaaatggcggccggcgcgtcggaacagggcgggaggcggcggcgcgtgggggcgggacggggaggggcgcggccagccgggcttctgcttccgcgaccccggcggtgcagggcgggtggagtcgcggagtagtccggcggggcgcgcgccgcgggtgggcggtgggtaaggacgttctccgcggagctcctaggcccggagcacgtgtcactggcttctctgccacccatgggcttgaaactattttaactgttcctagtccttatcccaagcctccaacattttaatctaagtggccgggacaaaattgaccaaaactagggggaacataggagtaattgctgagggatgacttgattgtactacgatgagatgaagggttaagatttcttccccatcaccagtagcctccctagctggtgtccctgtctccatactctcctgaatccaccttccactattgtccgctggccctttccagcctgtaaactggacagttttcattcctctgcctaacatccagtcgtgtcgaaattcgccaccatggctgctcctccagaacccggcgagcctgaggaacggaaggccagctgcaccagcctgcacctgagctactggaagtccctgaagctgctgggctttctggatgtggaaaacaccccttgcgcccggcacagcatcctgtatggcagcctgggaagcgtggtggccggcttcggccatttcctgttcacaagcagaattagaaggtcctgcgacgtgggcgtgggaggattcatcctggtcacactgggctgctggttccactgtagatacaactacgccaagcagagaatccaggagcggatcgccagagaggaaatcaagaaaaaaatcctgtacgagggcacccacctcgaccccgagagaaagcacaacggctcttctagcaattgaggtaccacggggtgaactacgcctgaggatcatcgatagatctctagtttaaacgggcccctcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgt

SEQ ID NO: 16 - DNA, 5694 bp, synthetic construct
  source 1..5694: mol_type = other DNA
aactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagaattcgcccttaagctagctagaccggtcaatgtctactctagcaaattttcaaagtcataataaaaataaaataggatgactcggtggtgttttagctggtacttttccagttttccttttacaacataatcaacaatgacttcaaaaggatttccccctccttcatggaatattactaccttcaacacggcctttcagatccggagttcaaagcatagttagcattataatttcagcagagaatccagaattggcaggatatcaccagtagggtgactttttttcttttccctagcaaacagtagagcagtatgtagtttctccccccgcccagacggagtttcgctctttctcccaggctggagtacagtaaaaaacctcaaaaaaaaaaatgtgacacataaatacacaaccttaactgtgtattttactgggattgtagaaaggtatcaaatgggcatgcaagtaaggtccttacggagtaagaaaacgcccaatgaacagtacttaatatgaattcgacatcgttaacactgagcgcttgctgtgtgccagcgcgcagcataattcgaagcagcatgactttgcacaatgacatattctacaagtccagtttcctaaggagtgcccttaggttacatataacctaaacgtgaaatgtagaagtacataaaggaggcataaagttaaaggggaaaaattataaaaattgcattgtattattttgatggctgaaaaatgggctcgaccctgcatgcacgcgttctccattgagctcgacaatatgcagtattttgtttcacgttatgtcgcagtcttagggaggagtgaaacgtggaacacgacttcacatcaaaaatgcacgttgcagaggcatttttcctgttaaagccacttagtaatgaaactataaaagggaggaagtggggtgggcgtgggagccccgtttgcgcaaggcaggcgcgggggactaaatggctgctggtgcagagtggggctgccccggcaccaagggctcgtcccgtggccggaaggacaaaggacacttaggggacgagggccgccgagccagcgcggagcgccttccgggtcggtgggctggggcgggctgggggctggggccgggccgacccccgcggtggggcccaccccgcaggacaaagcagcggccccgcccctcggcgcgcgcccgaggctcgtgcgtgtgagcacgcgcgccggagacggttagaaaagaaagtgtgacccgttcgacggaacaaaggacatgacagcccggccccgtgcggccactgcggagcgttaggaacattccctaaaatggcggccggcgcgtcggaacagggcgggaggcggcggcgcgtgggggcgggacggggaggggcgcggccagccgggcttctgcttccgcgaccccggcggtgcagggcgggtggagtcgcggagtagtccggcggggcgcgcgccgcgggtgggcggtgggtaaggacgttctccgcggagctcctaggcccggagcacgtgtcactggcttctctgccacccatgggcttgaaactattttaactgttcctagtccttatcccaagcctccaacattttaatctaagtggccgggacaaaattgaccaaaactagggggaacataggagtaattgctgagggatgacttgattgtactacgatgagatgaagggttaagatttcttccccatcaccagtagcctccctagctggtgtccctgtctccatactctcctgaatccaccttccactattgtccgctggccctttccagcctgtaaactggacagttttcattcctctgcctaacatccagtcgtgtcgaaattcgccaccatggccgctccccccgagccaggcgagcctgaggaaagaaagtccctgaagctgctgggcttcctggacgtggaaaacaccccttgcgccagacacagcatcctgtatggcagcctgggatctgtggtcgccggcttcggccacttcctgttcaccagcagaatccggagaagctgcgacgtgggcgtgggaggctttatcctggtgacactgggatgttggttccactgcagatacaactacgccaagcagcggattcaggagagaatcgcccgcgaggaaatcaagaaaaagatcctctacgagggcacccacctggatcctgaacggaaacataatggctctagcagcaactgaggtaccacggggtgaactacgcctgaggatcatcgatagatctctagtttaaacgggcccctcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgt

(19) United States
(12) Patent Application Publication
(10) Pub. No.: US 2025/0319209 A1
(43) Pub. Date: Oct. 16, 2025

(54) RAAV VECTOR FOR THE TREATMENT OF COX20 DEFICIENCY

(71) Applicant: University of Massachusetts, Westborough, MA (US)

(72) Inventors: Dominic Gessler, Worcester, MA (US); Guangping Gao, Worcester, MA (US); Peiyi Guo, Worcester, MA (US)

(73) Assignee: University of Massachusetts, Westborough, MA (US)

(21) Appl. No.: 19/250,220

(22) Filed: Jun. 26, 2025

Related U.S. Application Data
(63) Continuation of application No. PCT/US2024/024710, filed on Apr. 16, 2024.
(60) Provisional application No. 63/496,429, filed on Apr. 17, 2023.

(51) Int. Cl. A61K 48/00; A61K 38/17; A61P 3/00; C12N 15/86

(52) U.S. Cl. CPC A61K 48/0058; A61K 38/1709; A61P 3/00; C12N 15/86; C12N 2750/14143; C12N 2750/14145

(57) ABSTRACT

Aspects of the disclosure provide compositions and methods for promoting expression of functional COX20 protein in a subject. In some embodiments, the disclosure provides methods of treating a subject having COX20 deficiency.

### RELATED APPLICATION

This application claims the benefit under 35 U.S.C. § 119 (e) of the filing date of U.S. Provisional Application No. 63/496,429, filed on Apr. 17, 2023, the entire contents of which is incorporated herein by reference.

### REFERENCE TO AN ELECTRONIC SEQUENCE LISTING

The content of the electronic sequence listing (U012070189WO00-SEQ-MSB xml; Size: 57,748 bytes, and Date of Creation Apr. 15, 2024) is herein incorporated by reference in its entirety.

### BACKGROUND

COX20 deficiency (i.e., mitochondrial complex IV deficiency, nuclear type 11=MC4DN11) is a rare, autosomal recessive, neurologic disease primarily affecting children. Patients with COX20 deficiency develop early onset hypotonia, ataxia, areflexia, dystonia, dysarthria, and sensory neuronopathy. There are currently no treatment options available other than supportive care.

### SUMMARY

Aspects of the disclosure relate to a gene replacement therapy to restore COX20 expression and function in subjects, primarily in the nervous system (e.g., central nervous system or peripheral nervous system), which is useful for alleviating disease symptoms associated with COX20 deficiency (e.g., elevated lactate production). According to some aspects, the disclosure provides compositions and methods for promoting expression of functional COX20 in a subject. In some aspects, the disclosure provides methods of treating a subject having COX20 deficiency.

Accordingly, in some aspects, the disclosure provides an isolated nucleic acid comprising a transgene encoding a Cytochrome C Oxidase Assembly Factor COX20 (COX20) protein, wherein the transgene comprises a nucleic acid sequence having at least 70%, 75%, 80%, 85%, 90%, 95%, 98%, 99%, or 100% identity to SEQ ID NOs: 1 or 2. In some embodiments, the COX20 protein comprises an amino acid sequence having at least 70%, 75%, 80%, 85%, 90%, 95%, 98%, 99%, or 100% identity to an amino acid sequence set forth in SEQ ID NOs: 3 or 4.

In some embodiments, the isolated nucleic acid further comprises a promoter. In some embodiments, the promoter is a constitutive promoter, an inducible promoter, or a tissue-specific promoter. In some embodiments, the promoter is a COX20 promoter, optionally a human COX20 promoter. In some embodiments, the COX20 promoter comprises a sequence having at least 70%, 75%, 80%, 85%, 90%, 95%, 98%, 99%, or 100% identity to any one of SEQ ID NOs: 9-12.

In some embodiments, the isolated nucleic acid further comprises at least one adeno-associated virus (AAV) inverted terminal repeat (ITR). In some embodiments, the at least one AAV ITR is an AAV2 ITR. In some embodiments, the at least one AAV ITR is a truncated ITR (AITR).

In some embodiments, the isolated nucleic acid comprises a sequence having at least 70%, 75%, 80%, 85%, 90%, 95%, 98%, 99%, or 100% identity to any one of SEQ ID NOs: 5-8 or 13-16.

In some aspects, the disclosure provides a recombinant adeno-associated virus (IAA V) comprising the isolated nucleic acid of any of the above paragraphs and at least one AAV capsid protein. In some embodiments, the at least one AAV capsid protein is an AAV1, AAV2, AAV3, AAV4. AAV5, AAV6, AAV7, AAV8, AAV9, AAV.PHP-Eb. AAV.rh 10 capsid protein, or a variant thereof.

In some aspects, the disclosure provides a vector comprising the isolated nucleic acid of any of the above paragraphs. In some embodiments, the vector is a plasmid or a viral vector. In some embodiments, the viral vector is an adenoviral vector, an adeno-associated virus vector, a lentiviral vector, a retroviral vector, or a Baculovirus vector.

In some aspects, the disclosure provides a recombinant adeno-associated virus (rAAV) comprising: (i) an isolated nucleic acid comprising a transgene encoding a Cytochrome C Oxidase Assembly Factor COX20 (COX20) protein, wherein the transgene comprises a nucleic acid sequence having at least 70%, 75%, 80%, 85%, 90%, 95%, 98%, 99%, or 100% identity to SEQ ID NOs: 1 or 2; and (ii) at least one AAV capsid protein. In some embodiments, the COX20 protein comprises an amino acid sequence having at least 70%, 75%, 80%, 85%, 90%, 95%, 98%, 99%, or 100% identity to an amino acid sequence set forth in SEQ ID NOs: 3 or 4.

In some embodiments, the rAAV is a self-complementary AAV (scAAV) or a single-stranded AAV (ssAAV).

In some embodiments, the at least one AAV capsid protein has a tropism for nervous system cells, optionally neuronal cells. In some embodiments, the at least one AAV capsid protein is an AAV1, AAV2, AAV3, AAV4, AAV5, AAV6, AAV7, AAV8, AAV9, AAV.PHP-Bb, AAV.h10 capsid protein, or a variant thereof.

In some aspects, the disclosure provides a composition comprising the isolated nucleic acid or the rAAV of any of the above paragraphs, and a pharmaceutically acceptable excipient.

In some aspects, the disclosure provides a host cell comprising the isolated nucleic acid or the rAAV of any of the above paragraphs. In some embodiments, the host cell is a bacterial cell, a mammalian cell, or an insect cell. In some embodiments, the mammalian cell is a human cell.

In some aspects, the disclosure provides a method for treating Cytochrome C Oxidase Assembly Factor COX20 (COX20) deficiency in a subject, the method comprising administering to the subject the isolated nucleic acid or the rAAV of any of the above paragraphs. In some embodiments, the administration results in a decrease in a lactate level in the subject relative to the lactate level in the subject prior to the administration. In some aspects, the disclosure provides a method of decreasing a lactate level in a subject, the method comprising administering to the subject the isolated nucleic acid or the rAAV of any of the above paragraphs.

In some embodiments, the subject is a human. In some embodiments, the subject has one or more mutations in a COX20 gene.

In some embodiments, the administering is performed via an injection. In some embodiments, the injection comprises a systemic injection or an injection directly into the central nervous system of the subject.

In some aspects, the disclosure provides a method for preventing or treating Cytochrome C Oxidase Assembly Factor COX20 (COX20) deficiency in a subject, the method comprising administering to the subject an isolated nucleic acid comprising a transgene encoding a COX20 protein, wherein the transgene comprises a nucleic acid sequence having at least 70%, 75%, 80%, 85%, 90%, 95%, 98%, 99%, or 100% identity to SEQ ID NOs: 1 or 2. In some embodiments, the administration results in a decrease in a lactate level in the subject relative to the lactate level in the subject prior to the administration. In some aspects, the disclosure provides a method of decreasing a lactate level in a subject, the method comprising administering to the subject an isolated nucleic acid comprising a transgene encoding a Cytochrome C Oxidase Assembly Factor COX20 (COX20) protein, wherein the transgene comprises a nucleic acid sequence having at least 70%, 75%, 80%, 85%, 90%, 95%, 98%, 99%, or 100% identity to SEQ ID NOs: 1 or 2.

In some embodiments, the COX20 protein comprises an amino acid sequence having at least 700%, 75%, 80%, 85%, 90%, 95%, 98%, 99%, or 100% identity to an amino acid sequence set forth in SEQ ID NOs: 3 or 4.

In some embodiments, the isolated nucleic acid further comprises a promoter. In some embodiments, the promoter is a constitutive promoter, an inducible promoter, or a tissue-specific promoter. In some embodiments, the promoter is a COX20 promoter, optionally a human COX20 promoter. In some embodiments, the COX20 promoter comprises a sequence having at least 70%, 75%, 80%, 85%, 90%, 95%, 98%, 99%, or 100% identity to any one of SEQ ID NOs: 9-12.

In some embodiments, the isolated nucleic acid further comprises at least one adeno-associated virus (AAV) inverted terminal repeat (ITR). In some embodiments, the at least one AAV ITR is an AAV2 ITR. In some embodiments, the at least one AAV ITR is a truncated ITR (AITR).

In some embodiments, the subject is a human. In some embodiments, the subject has at least one mutation in a COX20 gene.

In some embodiments, the administering is performed via an injection. In some embodiments, the injection comprises a systemic injection or an injection directly into the central nervous system of the subject.

### BRIEF DESCRIPTION OF DRAWINGS

FIG. 1 shows fluorescence and bright-field microscopy images of HEK293 cells treated with water (control) or transfected with a vector encoding an enhanced green fluorescent protein (eGFP) operably linked to (i) cytomegalovirus (CMV) promoter, (ii) full-length human COX20 (Phcox20_full-cGFP), or (iii) partial human COX20 (Phcox20_partial-eGFP) promoter.

FIG. 2 shows immunofluorescence microscopy images of tissues (liver, muscle, heart, and kidney) from mice injected retro-orbitally (e.g., intravenously) with rAAVs comprising vectors encoding eGFP operably linked to a full-length human COX20 (Phcox20_full-eGFP) or a partial human COX20 (Phcox20_partial-eGFP) promoter.

FIG. 3 shows immunofluorescence microscopy images of central nervous system tissues (brain and spinal cord) from mice injected retro-orbitally (e.g., intravenously) with rAAVs comprising vectors encoding eGFP operably linked to a full-length human COX20 (Phcox20_full-eGFP) or a partial human COX20 (Pheox20_partial-eGFP) promoter.

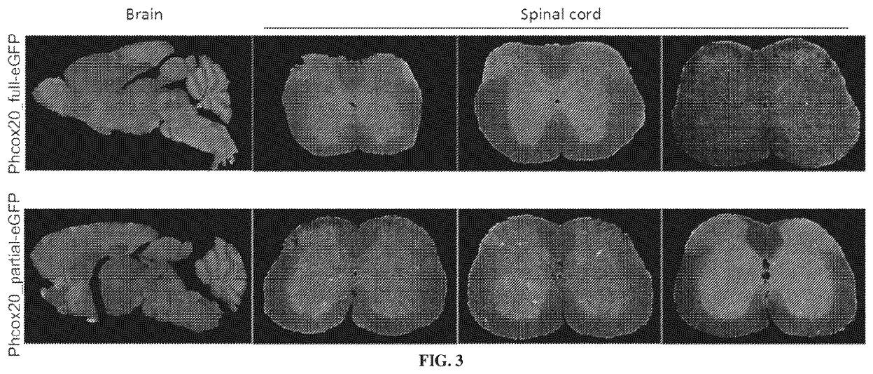

FIG. 4 shows a Western Blot analysis of eGFP expression in the livers of mice injected with rAAV's comprising vectors encoding eGFP operably linked to a full-length human COX20 (Full, left blot) or a partial human COX20 (Partial, right blot) promoter. rAAVs injected retro-orbitally are shown in lanes 7-9 of each blot. Lane 10 shows the livers from untreated control mice.

### DETAILED DESCRIPTION OF INVENTION

Aspects of the disclosure relate to compositions and methods for promoting expression of Cytochrome C Oxidase Assembly Factor COX20 (COX20) protein in a cell or subject. The disclosure is based, in part, on methods for treating a subject having COX20 deficiency.

### Cytochrome C Oxidase Assembly Factor COX20 (COX20) and COX20 Deficiency

Aspects of the disclosure relate to compositions (e.g., isolated nucleic acids, vectors such as recombinant adeno-associated virus (rAAV) vectors, recombinant adeno-associated viruses (rAAVs), etc.) that encode a Cytochrome C Oxidase Assembly Factor COX20 (COX20) protein. COX20 is a ubiquitously expressed protein required for correct assembly and function of mitochondrial Complex IV in the inner mitochondrial membrane (IMM). Complex IV is the fast of the four complexes in the oxidative phosphorylation cascade. It is responsible for moving H+ ions from the mitochondrial matrix to the intermembrane space (IMS) to generate an electrochemical gradient across the IMM, which is required for ATP Synthase to convert adenosine diphosphate (ADP) to adenosine triphosphate (ATP). Complex IV comprises at least 13 proteins, which require a plethora of other chaperone proteins to properly assemble. COX20 is one of such chaperone proteins.

The gene encoding for COX20, COX20, is located on chromosome 1 in humans and mice, and encodes for two isoforms: COX20-201 (130 amino acids) and COX20-203 (118 amino acids). In some embodiments, the COX20-201 isoform comprises the amino acid sequence set forth in SEQ ID NO: 3. In some embodiments, the COX20-203 isoform comprises the amino acid sequence set forth in SEQ ID NO: 4.

Loss of COX20 expression and/or function causes COX20 deficiency, a rare, autosomal recessive disease leading to early onset hypotonia, ataxia, areflexia, dystonia, dysarthria, and sensory neuronopathy. Interestingly, COX20 deficiency does not present with severe cognitive or intellectual disabilities, indicating primary defects in the peripheral nervous system and a primary degeneration of sensory neurons in the dorsal root ganglia (DRG).

### Isolated Nucleic Acids

In some aspects, the disclosure provides a nucleic acid comprising at least one transgene operably linked to a promoter, wherein the transgene encodes COX20 protein (e.g., a COX20-201 isoform, and/or a COX20-203 isoform). The COX20 gene may encode an mRNA having the nucleotide sequence of NM_001312871.1, NM_001312872.1, NM_001312873.1, NM_001312874.1, or NM_198076.6. The COX20 gene may encode a protein having the amino acid sequence NP_001299800.1, NP_001299801.1, NP_001299802.1, NP_001299803.1, or NP_932342.1. In some embodiments, the COX20 gene is codon-optimized (e.g., codon-optimized for expression in mammalian cells, such as human cells). Sequences corresponding to GenBank accession numbers described in the disclosure are incorporated herein by reference in their entirety.

In some embodiments, an isolated nucleic acid encoding a COX20 protein comprises the sequence set forth in SEQ ID NOs: 1 or 2. In some embodiments, the nucleic acid sequence encoding COX20 comprises at least 80%, at least 85%, at least 90%, at least 95%, at least 96%, at least 97%, at least 98%, or at least 99% sequence identity to the sequence set forth in SEQ ID NOs: 1 or 2. In some embodiments, the nucleic acid sequence encoding COX20 comprises up to 20 nucleotides that are different from the COX20 gene sequence set forth in SEQ ID NOs: 1 or 2. In some embodiments, the COX20 gene comprises more than 20 nucleotides that are different from the COX20 gene set forth in SEQ ID NOs: 1 or 2.

In some embodiments, the nucleic acid sequence encoding COX20 comprises insertions relative to SEQ ID NOs: 1 or 2. In some embodiments, the nucleic acid sequence encoding COX20 comprises insertions relative to SEQ ID NOs: 1 or 2 that do not introduce a frameshift mutation. In some embodiments, an insertion in the nucleic acid sequence relative to SEQ ID NOs: 1 or 2 involves the insertion of multiples of 3 nucleotides (e.g., 3, 6, 9, 12, 15, 18, etc.). In some embodiments, an insertion in the nucleic acid sequence relative to SEQ ID NOs: 1 or 2 leads to an increase in the total number of amino acid residues in the resultant COX20 protein (e.g., an increase of 1-3, 1-5, 3-10, 5-10, 5-15, or 10-20 amino acid residues).

In some embodiments, the nucleic acid sequence encoding COX20 comprises deletions relative to SEQ ID NOs: 1 or 2. In some embodiments, the nucleic acid sequence encoding COX20 comprises deletions relative to SEQ ID NOs: 1 or 2 that do not introduce a frameshift mutation. In some embodiments, a deletion in the nucleic acid sequence relative to SEQ ID NOs: 1 or 2 involves the deletion of multiples of 3 nucleotides (e.g., 3, 6, 9, 12, 15, 18, etc.). In some embodiments, a deletion in the nucleic acid sequence relative to SEQ ID NOs: 1 or 2 leads to a decrease in the total number of amino acid residues in the resultant COX20 protein (e.g., a decrease of 1-3, 1-5, 3-10, 5-10, 5-15, or 10-20 amino acid residues).

In some embodiments, the nucleic acid sequence encoding COX20 is a codon-optimized sequence (e.g., codon-optimized for expression in mammalian cells). In some embodiments, a codon-optimized sequence encoding COX20 comprises reduced GC content relative to a wild-type sequence that has not been codon-optimized. In some embodiments, a codon-optimized sequence encoding COX20 comprises a 1-5%, 3-5%, 3-10%, 5-10%, 5-15%, 10-20%, 15-30%, 20-40%, 25-50%, or 30-60% % reduction in GC content relative to a wild-type sequence that has not been codon-optimized. In some embodiments, a codon-optimized sequence encoding COX20 comprises fewer guanine and/or cytosine nucleobases relative to a wild-type sequence that has not been codon-optimized. In some embodiments, a codon-optimized sequence encoding COX20 comprises 1-5, 3-5, 3-10, 5-10, 5-15, 10-20, 15-30, 20-40, 25-50, or 30-60 fewer guanine and/or cytosine nucleobases relative to a wild-type sequence that has not been codon-optimized. In some embodiments, a codon-optimized sequence encoding COX20 comprises fewer CpG dinucleotide islands relative to a wild-type sequence that has not been codon-optimized. In some embodiments, a codon-optimized sequence encoding COX20 comprises 1-3, 3-5, 3-10, 5-10, 5-15, 10-20, 15-30, 20-40, 25-50, or 30-60 fewer CpG dinucleotide islands relative to a wild-type sequence that has not been codon-optimized.

An isolated nucleic acid encoding a COX20 protein is generally operably linked to a promoter. As used herein, “operably linked” refers to a promoter that is linked to and promotes expression of a downstream transgene. In some embodiments, the promoter is a constitutive promoter, for example, a chicken beta-actin (CB) promoter, a retroviral Rous sarcoma virus (RSV) LTR promoter (optionally with the RSV enhancer region), a cytomegalovirus (CMV) promoter (optionally with the CMV enhancer region) [see. e.g., Boshart et al., Cell, 41:521-530 (1985)], a SV40 promoter, a dihydrofolate reductase promoter, a β-actin promoter, a phosphoglycerol kinase (PGK) promoter, or an EF1α promoter [Invitrogen]. In some embodiments, a promoter is an enhanced chicken β-actin promoter. In some embodiments, a promoter is a U6 promoter. In some embodiments, the promoter is a CB6 promoter. In some embodiments, the promoter is a JeT promoter.

In some embodiments, a promoter is an inducible promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zine-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA. 93:3346-3351 (1996)), the tetracycline-repressible system (Gossen et al., Proc. Natl. Acad. Sci. USA. 89:5547-5551 (1992), the tetracycline-inducible system (Gossen et al., Science, 268:1766-1769 (1995), see also Harvey et al., Con, Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin inducible system (Magari et al., J. Clin. Invest., 100:2865-2872 (1997). Still other types of inducible promoters which may be useful in this context are those which are regulated by a specific physiological state, e.g., temperature, acute phase, a particular differentiation state of the cell, or in replicating cells only. In another embodiment, the native promoter for the transgene (e.g., COX20) will be used. The native promoter may be preferred when it is desired that expression of the transgene should mimic the expression of a native wild-type COX20 gene (e.g., a non-mutated COX20 gene). The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, a native promoter (e.g., a COX20) promoter may be modified for enhanced gene expression. In some embodiments, a COX20 promoter is a human COX20 promoter or is derived from a human COX20 promoter. In some embodiments, a COX20 promoter comprises a sequence having at least 70%, 75%, 80%, 85%, 90%, 91%, 92%, 93%, 94%, 95%, 96%, 97%, 98%, 99%, or 100% identity to any one of SEQ ID NOs: 9-12. In some embodiments, a COX20 promoter comprises a sequence having at least 70%, 75%, 80%, 85%, 90%, 91%, 92%, 93%, 94%, 95%, 96%, 97%, 98%, 99%, or 100% identity to SEQ ID NO: 9. In some embodiments, a COX20 promoter comprises a sequence having at least 70%, 75%, 809, 85%, 90%, 91%, 92%, 93%, 94%, 95%, 96%, 97%, 98%, 99%, or 100% identity to SEQ ID NO: 10. In some embodiments, a COX20 promoter comprises a sequence having at least 70%, 75%, 80%, 85%, 90%, 91%, 92%, 93%, 94%, 95%, 96%, 97%, 98%, 99%, or 100% identity to SEQ ID NO: 11. In some embodiments, a COX20 promoter comprises a sequence having at least 70%, 75%, 80%, 85%, 90%, 91%, 92%, 93%, 94%, 95%, 96%, 97%, 98%, 99%, or 100% identity to SEQ ID NO: 12. In some embodiments, a COX20 promoter is 50-350, 100-500, 50-200, 50-150, 200-500, 250-500, 300-600, 400-800, 500-1000, 500-1500, or 100-3000 nucleotides in length. In some embodiments, a COX20 promoter comprises a sequence having at least 70%, 75%, 80%, 85%, 90%, 91%, 92%, 93%, 94%, 95%, 96%, 97%, 98%, 99%, or 100% identity to a segment of SEQ ID NO: 9, wherein the segment of SEQ ID NO: 9 is 50-350, 100-500, 50-200, 50-150, 200-500, 250-500, 300-600, 400-800, 500-1000, 500-1500, or 100-2821 nucleotides in length. In some embodiments, a COX20 promoter comprises a sequence having at least 70%, 75%, 80%, 85%, 90%, 91%, 92%, 93%, 94%, 95%, 96%, 97%, 98%, 99%, or 100% identity to a segment of SEQ ID NO: 10, wherein the segment of SEQ ID NO: 10 is 50-350, 100-500, 50-200, 50-150, 200-500, 250-500, 300-600, 400-800, 500-1000, 500-1500, or 100-1818 nucleotides in length. In some embodiments, a COX20 promoter comprises a sequence having at least 70%, 75%, 80%, 85%, 90%, 91%, 92%, 93%, 94%, 95%, 96%, 97%, 98%, 99%, or 100% identity to a segment of SEQ ID NO: 11, wherein the segment of SEQ ID NO: 11 is 50-350, 50-200, 50-150, 50-100, 75-150, 100-350, or 100-200 nucleotides in length. In some embodiments, a COX20 promoter comprises a sequence having at least 70%, 75%, 80%, 85%, 90%, 91%, 92%, 93%, 94%, 95%, 96%, 97%, 98%, 99%, or 100% identity to a segment of SEQ ID NO: 12, wherein the segment of SEQ ID NO: 12 is 50-219, 50-200, 50-150, 50-100, 75-150, 100-219, or 100-200 nucleotides in length.

In some embodiments, the promoter drives transgene expression in neuronal tissues. In some embodiments, the disclosure provides a nucleic acid operably comprising a tissue-specific promoter operably linked to a transgene. As used herein. “tissue-specific promoter” refers to a promoter that preferentially regulates (e.g., drives or up-regulates) gene expression in a particular cell type relative to other cell types. A cell-type-specific promoter can be specific for any cell type, such as central nervous system (CNS) cells, peripheral nervous system cells, liver cells (e.g., hepatocytes), heart cells, muscle cells, etc.

Further examples of tissue-specific promoters include but are not limited to a liver-specific thyroxin binding globulin (TBG) promoter, an insulin promoter, a creatine kinase (MCK) promoter, a α-myosin heavy chain (a-MHC) promoter, or a cardiac Troponin T (cTnT) promoter. Other exemplary promoters include Beta-actin promoter, hepatitis B virus core promoter. Sandig et al., Gene Ther., 3:1002-9 (1996); alpha-fetoprotein (AFP) promoter, Arbuthnot et al., Hum. Gene Ther., 7:1503-14 (1996), bone osteocalcin promoter (Stein et al., Mol. Biol, Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998), and the immunoglobulin heavy chain promoter, among others which will be apparent to the skilled artisan.

As used herein, the term “hybrid promoter” refers to a regulatory construct capable of driving transcription an RNA transcript (e.g., a transcript comprising encoded by a transgene) in which the construct comprises two or more regulatory elements artificially arranged. Typically, a hybrid promoter comprises at least one element that is a minimal promoter and at least one element having an enhancer sequence or an intronic, exonic, or UTR sequence comprising one or more transcriptional regulatory elements. In embodiments in which a hybrid promoter comprises an exonic, intronic, or UTR sequence, such sequence(s) may encode upstream portions of the RNA transcript while also containing regulatory elements that modulate (e.g., enhance) transcription of the transcript. In some embodiments, two or more elements of a hybrid promoter are from heterologous sources relative to one another. In some embodiments, a hybrid promoter comprises a first sequence from the chicken beta-actin promoter and a second sequence of the CMV enhancer. In some embodiments, a hybrid promoter comprises a first sequence from a chicken beta-actin promoter and a second sequence from an intron of a chicken-beta actin gene. In some embodiments, a hybrid promoter comprises a first sequence from the chicken beta-actin promoter fused to a CMV enhancer sequence and a sequence from an intron of the chicken-beta actin gene. In some embodiments, a hybrid promoter comprises a CB6 promoter. In some embodiments, a hybrid promoter comprises a JeT promoter.

In some aspects, the disclosure relates to isolated nucleic acids comprising a transgene (e.g., COX20) operably linked to a promoter via a chimeric intron. In some embodiments, a chimeric intron comprises a nucleic acid sequence from a chicken beta-actin gene, for example a non-coding intronic sequence from intron I of the chicken beta-actin gene. In some embodiments, the intronic sequence of the chicken beta-actin gene ranges from about 50 to about 150 nucleotides in length (e.g., any length between 50 and 150 nucleotides, inclusive). In some embodiments, the intronic sequence of the chicken beta-actin gene ranges from about 100 to 120 (e.g., 100, 101, 102, 103, 104, 105, 106, 107, 108, 109, 110, 111, 112, 113, 114, 115, 116, 117, 118, 119, or 120) nucleotides in length. In some embodiments, a chimeric intron is adjacent to one or more untranslated sequences (e.g., an untranslated sequence located between the promoter sequence and the chimeric intron sequence and/or an untranslated sequence located between the chimeric intron and the first codon of the transgene sequence). In some embodiments, each of the one or more untranslated sequences are non-coding sequences from a rabbit beta-globulin gene (e.g., untranslated sequence from rabbit beta-globulin exon 1, exon 2, etc.).

In some embodiments, the rAAV comprises a posttranscriptional response element. As used herein, the term “posttranscriptional response element” refers to a nucleic acid sequence that, when transcribed, adopts a tertiary structure that enhances expression of a gene. Examples of posttranscriptional regulatory elements include, but are not limited to, woodchuck hepatitis virus posttranscriptional regulatory element (WPRE), mouse RNA transport element (RTE), constitutive transport element (CTE) of the simian retrovirus type 1 (SRV-1), the CTE from the Mason-Pfizer monkey virus (MPMV), and the 5′ untranslated region of the human heat shock protein 70 (Hsp70 5′UTR). In some embodiments, the rAAV vector comprises a woodchuck hepatitis virus posttranscriptional regulatory element (WPRE).

In some embodiments, the vector further comprises conventional control elements which are operably linked with elements of the transgene in a manner that permits its transcription, translation and/or expression in a cell transfected with the vector or infected with the virus produced by the disclosure. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A number of expression control sequences, including promoters which are native, constitutive, inducible and/or tissue-specific, are known in the art and may be utilized.

A polyadenylation sequence generally is inserted following the transgene sequences and optionally before a 3′ AAV ITR sequence. A rAAV construct useful in the disclosure may also contain an intron, desirably located between the promoter/enhancer sequence and the transgene. One possible intron sequence is derived from SV-40 and is referred to as the SV-40 T intron sequence. Another vector element that may be used is an internal ribosome entry site (IRES). An TRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989].

A “nucleic acid” sequence refers to a DNA or RNA sequence. In some embodiments, the term nucleic acid captures sequences that include any of the known base analogues of DNA and RNA such as, but not limited to 4-acetylcytosine, 8-hydroxy-N6-methyladenosine, aziridinyleytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2˜ thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, the disclosure relates to an isolated nucleic acid encoding a COX20 protein, or a protein having substantial homology to a COX20 protein. In some embodiments, a protein having substantial homology to a COX20 protein is at least 50%, 60%, 70%, 80%, 90%, 95%, or 99% identical to the amino acid sequence set forth in SEQ ID NO: 3 or 4. In some embodiments, a protein having substantial homology to a COX20 protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequences set forth in SEQ ID NOs: 3 or 4.

“Homology” refers to the percent identity between two polynucleotides or two polypeptide moieties. The term “substantial homology”, when referring to a nucleic acid or fragment thereof, indicates that, when optimally aligned with appropriate nucleic acid insertions or deletions with another nucleic acid (or its complementary strand), there is nucleotide sequence identity in about 90% to 100% of the aligned sequences. When referring to a polypeptide, or fragment thereof, the term “substantial homology” indicates that, when optimally aligned with appropriate gaps, insertions, or deletions with another polypeptide, there is amino acid identity in about 90% to 100% of the aligned sequences. The term “highly conserved” means at least 80% identity, preferably at least 90% identity, and more preferably over 97% identity. In some cases, “highly conserved” may refer to 100% identity. Identity is readily determined by one of skill in the art by, for example, the use of algorithms and computer programs known by those of skill in the art.

As used herein, alignments between sequences of nucleic acids or polypeptides are performed using any of a variety of publicly or commercially available Multiple Sequence Alignment programs, such as “Clustal W”, accessible through Web Servers on the internet. Alternatively, Vector NTI utilities may also be used. There are also a number of algorithms known in the art that can be used to measure nucleotide sequence identity, including those contained in the programs described above. As another example, polynucleotide sequences can be compared using BLASTN, which provides alignments and percent sequence identity of the regions of the best overlap between the query and search sequences. Similar programs are available for the comparison of amino acid sequences, e.g., the “Clustal X” program, BLASTP. Typically, any of these programs are used at default settings, although one of skill in the art can alter these settings as needed. Alternatively, one of skill in the art can utilize another algorithm or computer program that provides at least the level of identity or alignment as that provided by the referenced algorithms and programs. Alignments may be used to identify corresponding amino acids between two peptides or proteins. A “corresponding amino acid” is an amino acid of a protein or peptide sequence that has been aligned with an amino acid of another protein or peptide sequence. Corresponding amino acids may be identical or non-identical. A corresponding amino acid that is a non-identical amino acid may be referred to as a variant amino acid.

Alternatively, for nucleic acids homology can be determined by hybridization of polynucleotides under conditions that form stable duplexes between homologous regions, followed by digestion with single-stranded-specific nuclease(s), and size determination of the digested fragments. DNA sequences that are substantially homologous can be identified, for example, in a Southern hybridization experiment under conditions as defined for that particular system.

Mutations contemplated herein, with respect to an amino acid sequence, include, without limitation, substitutions, deletions, and additions. An amino acid “substitution” is a change in a single amino acid relative to a reference amino acid sequence. An amino acid substitution may result in a change in charge of the side chain of the amino acid position (e.g., from negatively charged to positively charged). In some embodiments, an amino acid substitution results in a change in polarity or hydrophobicity of the side chain of the amino acid position. In some embodiments, an amino acid substitution is a conservative substitution (e.g., a change from valine to alanine). In some embodiments, an amino acid substitution results in a different amino acid at that position that has an “equivalent” charge, polarity, and or chemical class (defined by the amino acid side chain).

In some embodiments, proteins and nucleic acids of the disclosure are isolated. As used herein, the term “isolated” means artificially obtained or produced. As used herein with respect to nucleic acids, the term “isolated” generally means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one that is readily manipulable by recombinant DNA techniques well known in the art. Thos, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” generally refers to a protein or peptide that has been artificially obtained or produced (e.g., by chemical synthesis, by recombinant DNA technology, etc.).

It should be appreciated that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

Recombinant AAVs (rAAVs)

The isolated nucleic acids of the disclosure may be recombinant adeno-associated viruses (rAAVs) vectors. rAAV vectors of the disclosure are typically composed of, at a minimum, a transgene and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, which encodes a polypeptide, protein, functional RNA molecule or other gene product, of interest. The nucleic acid coding sequence is operatively linked to regulatory components in a manner which permits transgene transcription, translation, and/or expression in a cell of a target tissue.

In some embodiments, an isolated nucleic acid as described by the disclosure comprises a region (e.g., a first region) comprising a first adeno-associated virus (AAV) inverted terminal repeat (ITR), or a variant thereof and a second region comprising a transgene encoding COX20. The isolated nucleic acid (e.g., the recombinant AAV vector) may be packaged into a capsid protein and administered to a subject and/or delivered to a selected target cell. The transgene may also comprise a region encoding, for example, a protein and/or an expression control sequence (e.g., a poly-A tail), as described elsewhere in the disclosure.

The instant disclosure provides a vector comprising a single, cis-acting wild-type ITR. In some embodiments, the ITR is a 5′ ITR. In some embodiments, the ITR is a 3′ ITR. Generally, ITR sequences are about 145 bp in length. Preferably, substantially the entire sequences encoding the ITR(s) is used in the molecule, although some degree of minor modification of these sequences is permissible. In some embodiments, an ITR may be mutated at its terminal resolution site (TR), which inhibits replication at the vector terminus where the TR has been mutated and results in the formation of a self-complementary AAV. Another example of such a molecule employed in the present disclosure is a “cis-acting” plasmid containing the transgene, in which the selected transgene sequence and associated regulatory elements are flanked by the 5′ AAV ITR sequence and a 3′ hairpin-forming RNA sequence. AAV ITR sequences may be obtained from any known AAV, including presently identified mammalian AAV types. In some embodiments, an ITR sequence is an AAV2, AAV3, AAV4, AAV5, AAV6, AAV7, AAV8, AAVrh8, AAV9, AAV10, and/or AAVsrh10 ITR sequence.

In some embodiments, a rAAV vector comprises a nucleic acid sequence encoding a COX20 protein or a portion thereof. In some embodiments, an rAAV vector comprises the sequence set forth in any one of SEQ ID NOs: 5-8 or 13-16.

In some embodiments, the rAAV vector is a self-complementary vector that comprises a nucleic acid sequence encoding a COX20 protein or portion thereof.

The isolated nucleic acids and/or rAAVs of the present disclosure may be modified and/or selected to enhance the targeting of the isolated nucleic acids and/or rAAVs to a target tissue (e.g., CNS). Non-limiting methods of modifications and/or selections include AAV capsid serotypes (e.g., AAV9), tissue-specific promoters, and/or targeting peptides. In some embodiments, the isolated nucleic acids and rAAVs of the present disclosure comprise AAV capsid serotypes with enhanced targeting to CNS tissues (e.g., AAV9). In some embodiments, the isolated nucleic acids and rAAVs of the present disclosure comprise tissue-specific promoters. In some embodiments, the isolated nucleic acids and rAAVs of the present disclosure comprise AAV capsid serotypes with enhanced targeting to CNS tissues and tissue-specific promoters.

In some aspects, the disclosure provides isolated AAVs. As used herein with respect to AAVs, the term “isolated” refers to an AAV that has been artificially obtained or produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected. In some embodiments, the rAAV comprises an AAV1, AAV2, AAV3, AAV4, AAV5, AAV6, AAV7, AAV8, AAVrh8, AAV9, AAV10, AAV.rh10, or AAV.PHPB capsid protein, or a protein having substantial homology thereto. In some embodiments, the rAAV comprises an AAV9 capsid protein. In some embodiments, the rAAV comprises an AAVPHP.B capsid protein.

In some embodiments, the rAAVs of the disclosure are pseudotyped rAAVs. Pseudotyping is the process of producing viruses or viral vectors in combination with foreign viral envelope proteins. The result is a pseudotyped virus particle. With this method, the foreign viral envelope proteins can be used to alter host tropism or an increased/decreased stability of the virus particles. In some aspects, a pseudotyped rAAV comprises nucleic acids from two or more different AAVs, wherein the nucleic acid from one AAV encodes a capsid protein and the nucleic acid of at least one other AAV encodes other viral proteins and/or the viral genome. In some embodiments, a pseudotyped IAAV refers to an AAV comprising an inverted terminal repeats (ITRs) of one AAV serotype and a capsid protein of a different AAV serotype. For example, a pseudotyped AAV vector containing the ITRs of serotype X encapsidated with the proteins of Y will be designated as AAVX/Y (e.g., AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

Methods for obtaining recombinant AA Vs having a desired capsid protein are well known in the art. (See, for example, US Patent Application Publication Number US 2003/0138772, the contents of which are incorporated herein by reference in their entirety). Typically the methods involve culturing a host cell which contains a nucleic acid sequence encoding an AAV capsid protein or fragment thereof; a functional rep gene; a recombinant AAV vector composed of, AAV inverted terminal repeats (ITRs) and a transgene; and sufficient helper functions to permit packaging of the recombinant AAV vector into the AAV capsid proteins. Typically, capsid proteins are structural proteins encoded by the cap gene of an AAV. In some embodiments, AA Vs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, capsid proteins protect a viral genome, deliver a genome and/or interact with a host cell. In some aspects, capsid proteins deliver the viral genome to a host in a tissue specific manner.

In some embodiments, the AAV capsid protein is of an AAV serotype selected from the group consisting of AAV3. AAV4, AAV5, AAV6, AAV8, AAVrh8 AAV9. AAV10 and AAV.rh10. In some embodiments, the AAV capsid protein is of an AAV.rh8, AAV.rh10, or AAV.PHPB serotype. In some embodiments, the AAV capsid protein is of an AAV.rh8 serotype. In some embodiments, the AAV capsid protein is of an AAV9 serotype. In some embodiments, the AAV capsid protein is of an AAV.PHPB serotype.

In certain embodiments, the disclosure relates to rAAV vectors comprising artificial transcription elements. As used here, the term “artificial transcription element” refers, in some embodiments, to a synthetic sequence enabling the controlled transcription of DNA by an RNA polymerase to produce an RNA transcript. Transcriptionally active elements of the present disclosure are generally smaller than 500 bp, preferably smaller than 200 bp, more preferably smaller than 100, most preferably smaller than 50 bp. In some embodiments, an artificial transcription element comprises two or more nucleic acid sequences from transcriptionally active elements. Transcriptionally active elements are generally recognized in the art and include, for example, promoter, enhancer sequence, TATA box, G/C box, CCAAT box, specificity protein 1 (Sp1) binding site, Inr region, CRE (CAMP regulatory element), activating transcription factor 1 (ATFI) binding site, ATFI-CRE binding site, APBβ box, APBα box, CArG box, CCAC box and those disclosed by U.S. Pat. No. 6,346,415. Combinations of the foregoing transcriptionally active elements are also contemplated.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest.

In some embodiments, components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected components) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a nucleic acid comprising a sequence set forth in SEQ ID NOs: 1 or 2 that is operably linked to a promoter. In some embodiments, the disclosure relates to a composition comprising the host cell described above. In some embodiments, the composition comprising the host cell above further comprises a cryopreservative.

The recombinant AAV vector, rep sequences, cap sequences, and helper functions useful for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al. J. Virol., 70:520-532 (1993) and U.S. Pat. No. 5,478,745.

In some embodiments, recombinant AAV's may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV Helper function vector supports efficient AAV vector production without generating any detectable wild-type AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced through the cell membrane. A number of transfection techniques are generally known in the art. See, e.g., Graham et al. (1973) Virology, 52:456. Sambrook et al. (1989) Molecular Cloning, a laboratory manual, Cold Spring Harbor Laboratories, New York. Davis et al. (1986) Basic Methods in Molecular Biology. Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell (e.g., a non-human primate, rodent, or human cell). In some embodiments, the host cell is a mammalian cell, a yeast cell, a bacterial cell, an insect cell, a plant cell, or a fungal cell. A host cell may be used as a recipient of an AAV helper construct, an AAV minigene plasmid, an accessory function vector, or other transfer DNA associated with the production of recombinant. AAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the term “recombinant cell” refers to a cell into which an exogenous DNA segment, such as DNA segment that leads to the transcription of a biologically active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned.” “under control” of “under transcriptional control” means that the promoter is in the correct location and orientation in relation to the nucleic acid to control RNA polymerase initiation and expression of the gene. The term “expression vector or construct” means any type of genetic construct containing a nucleic acid in which part er all of the nucleic acid encoding sequence is capable of being transcribed. In some embodiments, expression includes transcription of the nucleic acid, for example, to generate a biologically active polypeptide product or inhibitory RNA (e.g., shRNA, miRNA, miRNA inhibitor) from a transcribed gene.

The foregoing methods for packaging recombinant vectors in desired AAV capsids to produce the rAAVs of the disclosure are not meant to be limiting and other suitable methods will be apparent to the skilled artisan.

rAAV Vector: Transgene Coding Sequences

The composition of the transgene sequence of the rAAV vector will depend upon the use to which the resulting vector will be put. For example, one type of transgene sequence includes a reporter sequence, which upon expression produces a detectable signal. In another example, the transgene encodes a therapeutic protein. In another example, the transgene encodes a protein that is intended to be used for research purposes. e.g., to create a somatic transgenic animal model harboring the transgene, e.g., to study the function of the transgene product. In another example, the transgene encodes a protein that is intended to be used to create an animal model of disease.

In some embodiments, the disclosure provides an rAAV comprising a transgene encoding COX20. Also contemplated herein are methods of treating COX20 deficiency by delivering a transgene to a subject using the rAAVs described herein. In some embodiments, the disclosure relates to a method for treating a COX20 deficiency, the method comprising administering a rAAV to a subject. In some embodiments, the rAAV comprises a hybrid promoter. In some embodiments, the rAAV comprises a chimeric intron. In some embodiments, the rAAV comprises an artificial transcription element. In some embodiments, the artificial transcription element comprises ATFI-CRE binding site. SPI binding site and TATA box. In some embodiments, the promoter, chimeric intron, or artificial transcription element is operably linked to a transgene. In some embodiments, the transgene encodes COX20.

In some embodiments, one or more bindings sites for one or more of miRNAs are incorporated in a transgene of a rAAV vector, to inhibit the expression of the transgene in one or more tissues of an subject harboring the transgene. The skilled artisan will appreciate that binding sites may be selected to control the expression of a transgene in a tissue specific manner. For example, binding sites for the liver-specific miR-122 may be incorporated into a transgene to inhibit expression of that transgene in the liver. The target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

rAAV Administration Methods

The rAAVs may be delivered to a subject in compositions according to any appropriate methods known in the art. The rAAV, preferably suspended in a physiologically compatible carrier (i.e., in a composition), may be administered to a subject, i.e., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the (AA Vs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue.

A method for delivering a transgene to CNS tissue in a subject may comprise administering a rAAV by a single route or by multiple routes. For example, delivering a transgene to CNS tissue in a subject may comprise administering to the subject, by intravenous administration, an effective amount of a rAAV that crosses the blood-brain barrier. Delivering a transgene to CNS tissue in a subject may comprise administering to the subject an effective amount of a rAAV by intrathecal administration or intracerebral administration, e.g., by intraventricular injection A method for delivering a transgene to CNS tissue in a subject may comprise co-administering of an effective amount of a rAAV by two different administration routes, e.g., by intrathecal administration and by intracerebral administration. Co-administration may be performed at approximately the same time, or different times.

The CNS tissue to be targeted may be selected from cortex, hippocampus, thalamus, hypothalamus, cerebellum, brain stem, cervical spinal cord, thoracic spinal cord, and lumbar spinal cord, for example. The administration route for targeting CNS tissue typically depends on the AAV serotype. For example, in certain instances where the AAV serotype is selected from AAVPHP.B, AAV1, AAV6, AAV6.2, AAV7, AAV8, AAV9, AAV.rh10, AAV.rh39, AAV.rh43 and CSp3, the administration route may be intravascular injection. In some instances, for example where the AAV serotype is selected from AAVPHP.B. AAV1, AAV2, AAV5, AAV6, AAV6.2. AAV7, AAV8, AAV9, AAV.th10, AAV.rh39, AAV.rh43 and CSp3, the administration route may be intrathecal and/or intracerebral injection.

Aspects of the disclosure relate to compositions comprising a recombinant AAV comprising at least one modified genetic regulatory sequence or element. In some embodiments, the composition further comprises a pharmaceutically acceptable carrier.

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAV's each having one or more different transgenes.

In some aspects, the disclosure relates to a composition (e.g., a pharmaceutical composition) comprising an rAAV comprising a nucleic acid encoding COX20.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., injection into the liver, skeletal muscle), oral, inhalation (including intranasal and intratracheal delivery), intraocular, intravenous, intramuscular, subcutaneous, intradermal, intratumoral, and other parental routes of administration. In some embodiments, an rAAV is delivered using any route of intravenous administration (e.g., retro-orbital injection). Routes of administration may be combined, if desired.

The dose of rAAV virions required to achieve a particular “therapeutic effect.” e.g., the units of dose in genome copies/per kilogram of body weight (OC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art.

An effective amount of an rAAV is an amount sufficient to target infect an animal, target a desired tissue. The effective amount will depend primarily on factors such as the species, age, weight, health of the subject, and the tissue to be targeted, and may thus vary among animal and tissue. For example, an effective amount of the rAAV is generally in the range from about 1 ml to about 100 ml of solution containing from about 10⁶ to 10¹⁶ genome copies (e.g., from 1×10⁶ to 1×10¹⁶, inclusive). In some cases, a dosage between about 10¹¹ to 10¹² rAAV genome copies is appropriate. In some embodiments, a dosage of between about 10¹¹ to 10¹² rAAV genome copies is appropriate. In some embodiments, a dosage of between about 10¹¹ to 10¹⁴ rAAV genome copies is appropriate. In some embodiments, a dosage of between about 10¹¹ to 10¹⁵ rAAV genome copies is appropriate. In some embodiments, a dosage of 4.68×10⁷ is appropriate. In some embodiments, a dosage of 4.68×10⁵ genome copies is appropriate. In some embodiments, a dosage of 4.68×10⁹ genome copies is appropriate. In some embodiments, a dosage of 1.17×10¹⁰ genome copies is appropriate. In some embodiments, a dosage of 2.34×10¹⁰ genome copies is appropriate. In some embodiments, a dosage of 3.20×10¹¹ genome copies is appropriate. In some embodiments, a dosage of 1.2×10¹³ genome copies is appropriate. In some embodiments, a dosage of about 1×10¹⁴ vector genome (vg) copies is appropriate.

In some aspects, the disclosure relates to the recognition that one potential side-effect for administering an AAV to a subject is an immune response in the subject to the AAV, including inflammation. In some embodiments, a subject is immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but, not limited to, rituximab, methylprednisolone, prednisolone, sirolimus, immunoglobulia injection, prednisone, methotrexate, and any combination thereof.

In some embodiments, methods described by disclosure further comprise the step inducing immunosuppression (e.g., administering one or more immunosuppressive agents) in a subject prior to the subject being administered an rAAV (e.g., an rAAV or pharmaceutical composition as described by the disclosure). In some embodiments, a subject is immunosuppressed (e.g., immunosuppression is induced in the subject) between about 30 days and about 0 days (e.g., any time between 30 days until administration of the rAAV, inclusive) prior to administration of the rAAV to the subject. In some embodiments, the subject is pre-treated with immune suppression (e.g., rituximab, sirolimus, and/or prednisone) for at least 7 days.

In some embodiments, immunosuppression of a subject maintained during and/or after administration of a rAAV or pharmaceutical composition. In some embodiments, a subject is immunosuppressed (e.g., administered one or more immunosuppressants) for between 1 day and 1 year after administration of the rAAV or pharmaceutical composition.

In some embodiments, rAAV compositions are formulated to reduce aggregation of AAV particles in the composition, particularly where high rAAV concentrations are present (e.g., ˜10¹³) GC/ml or more), Methods for reducing aggregation of rAAVs are well known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intrapancreatically, intranasally, parenterally, intravenously, intramuscularly, intrathecally, or orally, intraperitoneally, or by inhalation. In some embodiments, the administration modalities as described in U.S. Pat. Nos. 5,543,158, 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, using a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein. “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of ML Vs results in the formation of small unilamellar vesicles (SUV's) with diameters in the range of 200 to 500 Angstroms, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the disclosure relates to administration of one or more additional therapeutic agents to a subject who has been administered an rAAV or pharmaceutical composition as described herein.

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the disclosure relates to a kit for producing a rAAV, the kit comprising a container housing an isolated nucleic acid encoding a COX20 protein or a portion thereof. In some embodiments, the kit further comprises instructions for producing the rAAV. In some embodiments, the kit further comprises at least one container housing a recombinant AAV vector, wherein the recombinant AAV vector comprises a transgene.

In some embodiments, the disclosure relates to a kit comprising a container housing a recombinant AAV as described supra. In some embodiments, the kit further comprises a container housing a pharmaceutically acceptable carrier. For example, a kit may comprise one container housing a rAAV and a second container housing a buffer suitable for injection of the rAAV into a subject. In some embodiments, the container is a syringe.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on ox associated with packaging of the disclosure. Instructions also can include any oral or electronic instructions provided in any manner such that a user will clearly recognize that the instructions are to be associated with the kit, for example, audiovisual (e.g., videotape, DVD, etc.). Internet, and/or web-based communications, etc. The written instructions may be in a form prescribed by a governmental agency regulating the manufacture, use or sale of pharmaceuticals or biological products, which instructions can also reflect approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container. The kit may have one or more or all the components required to administer the agents to an animal, such as a syringe, topical application devices, or iv needle tubing and bag, particularly in the case of the kits for producing specific somatic animal models.

In some cases, the methods involve transfecting cells with total cellular DNAs isolated from the tissues that potentially harbor proviral AAV genomes at very low abundance and supplementing with helper virus function (e.g., adenovirus) to trigger and/or boost AAV rep and cap gene transcription in the transfected cell. In some cases, RNA from the transfected cells provides a template for RT-PCR amplification of eDNA and the detection of novel AAVs. In cases where cells are transfected with total cellular DNAs isolated from the tissues that potentially harbor proviral AAV genomes, it is often desirable to supplement the cells with factors that promote AAV gene transcription. For example, the cells may also be infected with a helper virus, such as an Adenovirus or a Herpes Virus. In a specific embodiment, the helper functions are provided by an adenovirus. The adenovirus may be a wild-type adenovirus, and may be of human or non-human origin, preferably non-human primate (NHP) origin. Similarly, adenoviruses known to infect non-human animals (e.g., chimpanzees, mouse) may also be employed in the methods of the disclosure (See, e.g., U.S. Pat. No. 6,083,716). In addition to wild-type adenoviruses, recombinant viruses, or non-viral vectors (e.g., plasmids, episomes, etc.) carrying the necessary helper functions may be utilized. Such recombinant viruses are known in the art and may be prepared according to published techniques. Seo. e.g., U.S. Pat. Nos. 5,871,982 and 6,251,677, which describe a hybrid Ad/AAV virus. A variety of adenovirus strains are available from the American Type Culture Collection, Manassas, Va., or available by request from a variety of commercial and institutional sources. Further, the sequences of many such strains are available from a variety of databases including. e.g., PubMed and GenBank.

Cells may also be transfected with a vector (e.g., helper vector) which provides helper functions to the AAV. The vector providing helper functions may provide adenovirus functions, including, e.g., E1a, E1b, E2a, E4ORF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

In some cases, a novel isolated capsid gene can be used to construct and package recombinant AAV vectors, using methods well known in the art, to determine functional characteristics associated with the novel capsid protein encoded by the gene. For example, novel isolated capsid genes can be used to construct and package recombinant AAV (rAAV) vectors comprising a reporter gene (e.g., B-Galactosidase, GFP, Luciferase, etc.). The rAAV vector can then be delivered to an animal (e.g., mouse) and the tissue targeting properties of the novel isolated capsid gene can be determined by examining the expression of the reporter gene in various tissues (e.g., heart, liver, kidneys) of the animal. Other methods for characterizing the novel isolated capsid genes are disclosed herein and still others are well known in the art.

The kit may have a variety of forms, such as a blister pouch, a shrink-wrapped pouch, a vacuum scalable pouch, a scalable thermoformed tray, or a similar pouch or tray form, with the accessories loosely packed within the pouch, one or more tabes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for detecting a latent AAV in a cell. In addition, kits of the disclosure may include, instructions, a negative and/or positive control, containers, diluents and buffers for the sample, sample preparation tubes and a printed or electronic table of reference AAV sequence for sequence comparisons.

### Methods of Treating COX20 Deficiency

Aspects of the present disclosure provide methods for treating COX20 deficiency. COX20 deficiency results, in some embodiments, from loss-of-function mutations in the COX20 gene. In some embodiments, gene replacement therapy is provided herein that is useful to restore COX20 function, primarily in the nervous system, which can alleviate the disease symptoms.

Accordingly, in some embodiments, the disclosure provides isolated nucleic acids, rAAVs, compositions, and methods useful in treating COX20 deficiency. In some embodiments, the isolated nucleic acids, rAAVs, compositions, and methods are for the treatment of COX20 deficiency. Methods for treating COX20 deficiency in a subject may comprise administering an isolated nucleic acid, rAAV, or composition of the present claims that comprises a transgene encoding COX20.

In some aspects, the disclosure provides a method of promoting expression of functional COX20 protein in a subject (e.g., in the central nervous system of the subject) comprising administering the isolated nucleic acids, rAAV's, or the compositions described herein to a subject having or suspected of having a disease or disorder associated with low levels of COX20 expression and/or function (e.g., COX20 deficiency). As used herein, a disease or disorder associated with low levels of COX20 expression and/or function is a disease or disorder in which a subject has at least 10%, at least 20%, at least 30%, at least 40%, at least 50%, at least 60%, at least 70%, at least 80%, or at least 90% lower levels of COX20 expression and/or function relative to a control subject (e.g., a healthy subject and/or an untreated subject).

In some embodiments, administering the isolated nucleic acids, the rAAVs, or the compositions described herein to a subject promotes expression of COX20 by between 2-fold and 100-fold (e.g., 2-fold, 5-fold, 10-fold, 20-fold, 50-fold, 75-fold, 100-fold, etc.) compared to a control subject. In some embodiments, administering the isolated nucleic acids, the rAAVs, or the compositions described herein to a subject promotes expression of COX20 in the CNS of a subject by between 2-fold and 100-fold (e.g., 2-fold, 5-fold, 10-fold, 20-fold, 50-fold, 75-fold, 100-fold, etc.) compared to a control subject. As used herein a “control” subject may refer to a subject that is not administered the isolated nucleic acids, the rAAVs, or the compositions described herein; or a healthy subject. In some embodiments, a control subject is the same subject that is administered the isolated nucleic acids, the rAAVs, or the compositions described herein (e.g., prior to the administration). In some embodiments, administering the isolated nucleic acids, the rAAVs, or the compositions described to a subject promotes expression of COX20 by 2-fold compared to a control. In some embodiments, administering the isolated nucleic acids, the rAAVs, or the compositions described to a subject promotes expression of COX20 by 100-fold compared to a control. In some embodiments, administering the isolated nucleic acids, the rAAV's, or the compositions described to a subject promotes expression of COX20 by 5-fold compared to a control. In some embodiments, administering the isolated nucleic acids, the rAAVs, or the compositions described to a subject promotes expression of COX20 by 10-fold compared to a control. In some embodiments, administering the isolated nucleic acids, the rAAVs, or the compositions described to a subject promotes expression of COX20 by 5-fold to 100-fold compared to control (e.g., 5-fold to 10-fold, 10-fold to 15-fold, 10-fold to 20-fold, 15-fold to 25-fold, 20-fold to 30-fold, 25-fold to 35-fold, 30-fold to 40-fold, 35-fold to 45-fold, 40-fold to 60-fold, 50-fold to 75-fold, 60-fold to 80-fold, 75-fold to 100-fold compared to a control).

In some embodiments, administering the isolated nucleic acids, the rAAVs, or the compositions described herein to a subject promotes expression of COX20 in a subject (e.g., promotes expression of COX20 in the CNS of a subject) by between a 5% and 200% increase (e.g., 5-50%, 25-75%, 50-100%, 75-125%, 100-200%, or 100-150% etc.) compared to a control subject. In some embodiments, administering a composition, an isolated nucleic acid, and/or an rAAV as disclosed herein to a cell (e.g., a cell in a subject) reduces lactate levels in the cell by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control (e.g., relative to the cell prior to administration). In some embodiments, administration of a composition, an isolated nucleic acid, and/or an rAAV as disclosed herein to a cell results in decreased lactate levels in the cell (e.g., decreased lactate levels by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 1.5 times, at least 2 times, at least 3 times, at least 4 times, at least 5 times, at least 10 times, at least 20 times, at least 50 times or at least 100 times) relative to the cell prior to administration.

In some embodiments, administering a composition, an isolated nucleic acid, and/or an rAAV as disclosed herein to a cell (e.g., a cell in a subject) reduces symptoms associated with COX20 deficiency (e.g., hypotonia (e.g., early onset hypotonia), ataxia, areflexia, dystonia, dysarthria, and/or sensory neuronopathy) by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control (e.g., relative to the cell prior to administration). In some embodiments, administration of a composition, an isolated nucleic acid, and/or an rAAV as disclosed herein to a cell (e.g., a cell in a subject) reduces symptoms associated with COX20 deficiency (e.g., hypotonia (e.g., early onset hypotonia), ataxia, areflexia, dystonia, dysarthria, and/or sensory neuronopathy) (e.g., decreased symptoms associated with COX20 deficiency by at least 5%, at least 10%, at least 15%, at least 20%, at least 25%, at least 30% %, at least 35%, at least 40%, at least 45%, at least 50%, at least 55%, at least 60%, at least 65%, at least, 70%, at least 75%, at least 80%, at least 85%, at least 90%, at least 95%, at least 100%, at least 1.5 times, at least 2 times, at least 3 times, at least 4 times, at least 5 times, at least 10 times, at least 20 times, at least 50 times or at least 100 times) relative to the cell prior to administration.

In some embodiments, administering a composition, an isolated nucleic acid, and/or an rAAV as disclosed herein to a cell (e.g., a cell in a subject) reduces lactate levels in the cell by at least 5%, 10%, 20%, 30%, 40%, 50%, 60%, 70%, 80%, 90%, 100%, or by at least 2-fold, at least 5-fold, at least 10-fold, at least 20-fold, at least 50-fold, at least 100-fold, or at least 1000-fold compared to a control (e.g., relative to the cell prior to administration). In some embodiments, administration of a composition, an isolated nucleic acid, and/or an rAAV as disclosed herein to a cell results in decreased lactate levels in the cell (e.g., decreased lactate levels by at least 5%, at least 10%, at least 15%, at least 20%, at least 25%, at least 30%, at least 35%, at least 40%, at least, 45%, at least 50%, at least 55%, at least 60%, at least 65%, at least 70%, at least 75%, at least 80%, at least 85%, at least 90%, at least 95%, at least 100%, at least 1.5 times, at least 2 times, at least 3 times, at least 4 times, at least 5 times, at least 10 times, at least 20 times, at least 50 times or at least 100 times) relative to the cell prior to administration.

In some aspects, the disclosure provides a method of treating a subject having a disease or disorder associated with low levels of COX20 expression and/or function (e.g., COX20 deficiency), the method comprising administering to the subject an effective amount of an rAAV comprising a capsid containing a nucleic acid engineered to express COX20 in the CNS of the subject.

As used herein, the term “treating” refers to the application or administration of a composition (e.g., an isolated nucleic acid or rAAV as described herein) to a subject who has a disease or disorder associated with low levels of COX20 expression and/or function (e.g., COX20 deficiency), with the purpose to cure, heal, alleviate, relieve, alter, remedy, ameliorate, improve, or affect the disorder, the symptom of the disease, or the predisposition toward a disease associated with prolonged oxidative stress.

Alleviating a disease associated with low levels of COX20 expression and/or function (e.g., COX20 deficiency) includes delaying the development or progression of the disease, or reducing disease severity. Alleviating the disease does not necessarily require curative results. As used therein, “delaying” the development of a disease (such as a disease associated with inflammation (e.g., microgliosis), demyelination, and/or death of synaptic neurons) means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a disease associated with low levels of COX20 expression and/or function (e.g., COX20 deficiency).

A subject may be a human, a mouse, a rat, a pig, a dog, a cat, or a non-human primate. In some embodiments, a subject has or is suspected of having a disease or disorder associated with low levels of COX20 expression or activity (e.g., COX20 deficiency). In some embodiments, a subject having a disease or disorder associated with low levels of COX20 expression or activity (e.g., COX20 deficiency) comprises at least one COX20 allele having a loss-of-function mutation (e.g., associated with COX20 deficiency). In some embodiments, a COX20 allele having a loss-of-function mutation (e.g., associated with COX20 deficiency) comprises a frameshift mutation, a splice site mutation, a missense mutation, a truncation mutation, or a nonsense mutation. A subject may have two COX20 alleles having the same loss-of-function mutations (homozygous state) or two COX20 alleles having different loss-of-function mutations (compound heterozygous state).

Aspects of the disclosure relate to methods of treating a subject having a COX20 deficiency. In some embodiments, the subject has one or more mutations in a COX20 gene. Mutations in COX20 genes are known, for example as described by Li et al. Front Neurol. 2022 May 16; 13:873943. doi: 10.3389/fneur 2022.873943. eCollection 2022. In some embodiments, a COX20 allele having a loss-of-function mutation comprises a homozygous c.154A-C mutation in a COX20 gene.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., to the central nervous system), oral, inhalation (including intranasal and intratracheal delivery), intraocular, intravenous, intramuscular, subcutaneous, intradermal, intratumoral, and other parental routes of administration. Routes of administration may be combined, if desired.

### EXAMPLES

### Example 1. rAAV For The Treatment Of COX20 Deficiency

Cytochrome C Oxidase Assembly Factor COX20 (COX20) deficiency (aka mitochondria complex IV deficiency, nuclear type 11=MC4DN11) is a rare, autosomal recessive, neurologic disease primarily affecting children. It belongs to a large group of mitochondrial diseases with its subgroup known as Complex IV (cytochrome c oxidase) deficiency. Overall, Complex IV deficiency is estimated to affect ˜ 1:35,000 births. Complex IV requires COX20 for its correct assembly and function in the inner mitochondrial layer (IMM). Although the prevalence of COX20 deficiency is currently unknown, case reports from around the world indicate that this condition affects every social and cultural group. It presents with neurologic symptoms such as ophthalmoplegia (reduced eye movement), muscular hypotonia, cerebellar ataxia (lack of voluntary coordination of movements), delayed speech development, dystonia, and neuropathy.

Complex IV deficiency forces cells to rely heavily on glycolysis and less on oxidative phosphorylation, leading to elevated blood lactate levels. The increase in lactate can be exacerbated with physical activity and measured in blood, serving as a possible biomarker. Limited information is available about the histopathologic characteristics of COX20 deficiency, likely due to the relatively new discovery of COX20 deficiency. Biopsies from human patients show substantially reduced numbers of large, myelinated axons in peripheral nerves and atrophic muscle fibers. Currently, only supportive treatment is available. This example describes development of gene therapies for COX20 deficiency.

Complex IV is the last of the four complexes in the oxidative phosphorylation cascade. It is located in the IMM and functions to move H+ ions from the mitochondrial matrix (MM) to the intermembrane space (IMS). The electrochemical gradient between the IMS and MM is needed for the ATP Synthase to generate ATP. Complex IV comprises at least 13 proteins which require a plethora of other proteins to properly assemble Complex IV (e.g., chaperone proteins). These chaperone proteins are essential for Complex IV assembly. It is of no surprise that this finely tuned system is susceptible to many forms of disruptions leading to Complex IV deficiency. COX20 is one chaperone needed for the correct assembly of Complex IV. The gene encoding COX20 is located on chromosome 1 in humans and mice and encodes two protein isoforms: COX20-201 (130 amino acids), and COX20-203 (118 amino acids). Currently, it is believed that COX20, along with Synthesis of Cytochrome C Oxidase 1 (SCO1) and 2 (SCO2), are needed to mature mitochondrially-encoded cytochrome C oxidase II (COX2) for its incorporation in Complex IV. This COX2 maturation is interrupted by loss-of-function mutations in COX20, eventually destabilizing Complex IV and its function. With the loss of Complex IV, mitochondria lose substantial ability to generate energy. To fill the energetic gap, cells must upregulate glycolysis (glucose metabolism without the final oxidative phosphorylation) with concomitant increase in lactate.

Recombinant AAV (rAAV) vectors encoding either COX20-201 or COX20-203 were produced. Briefly, the vectors include a promoter (e.g., a chicken beta-actin (CB) promoter or a COX20 promoter) operably linked to a nucleic acid sequence encoding the COX20 isoform, a polyA region, and flanking AAV2 inverted terminal repeats. The rAAV vectors may then packaged in an AAV capsid protein (e.g., an AAV2 capsid protein), for administration to a subject (e.g., a subject having a COX20 deficiency), for example by systemic injection.

### Example 2. Delivery of AAV Vectors Encoding COX20 Protein to Mammalian Cells

AAV vectors were designed to comprise either a COX20 isoform (COX20-201 or COX20-203) under the control of a chicken beta-actin (CB) promoter in mammalian cells. HEK293 cells were transfected with one of the following AAV vectors-(1) AAV comprising a nucleic acid encoding COX20-201 (SEQ ID NO: 1) connected to a C-terminal FLAG tag operably linked to CB promoter. (2) AAV comprising a nucleic acid encoding COX20-201 (SEQ ID NO: 1) operably linked to CB promoter. (3) AAV comprising a nucleic acid encoding COX20-203 (SEQ ID NO: 2) connected to a C-terminal FLAG tag operably linked to CB promoter, and (4) AAV comprising a nucleic acid encoding COX20-203 (SEQ ID NO: 2) operably linked to CB promoter. HEK293 cells treated with water were used as control.

Protein expression analysis confirmed that the HEK293 cells expressed the COX20 proteins when transfected with any one of the AAV vectors. Expression levels of COX20 were similar whether or not the FLAG tag was present at the C-terminus. Co-localization analysis demonstrated that all COX20 proteins co-localized with mitochondria. These data demonstrate that the COX20-201 and COX20-203 isoforms (encoded by SEQ ID NOs: 1 and 2, respectively) are capable of expression in mammalian cells following transfection of AAV vectors encoding such proteins.

### Example 3. AAV Vectors Driven by a Human COX20 Promoter

The chromosome region surrounding the human COX20 gene, located on human chromosome 1, was analyzed using the UCSC Genome Browser (genome.ucsc.edu) to derive endogenous human COX20 promoters. Two promoter-like signatures were identified as ENCODE Candidate Cis-Regulatory Elements (cCREs) (E1438691/prom (SEQ ID NO: 11) and E1438692/prom (SEQ ID NO: 12)). These cCREs were combined to generate a “full-length” human COX20 (hCOX20) promoter (SEQ ID NO: 9) and a “partial” hCOX20 promoter (SEQ ID NO: 10).

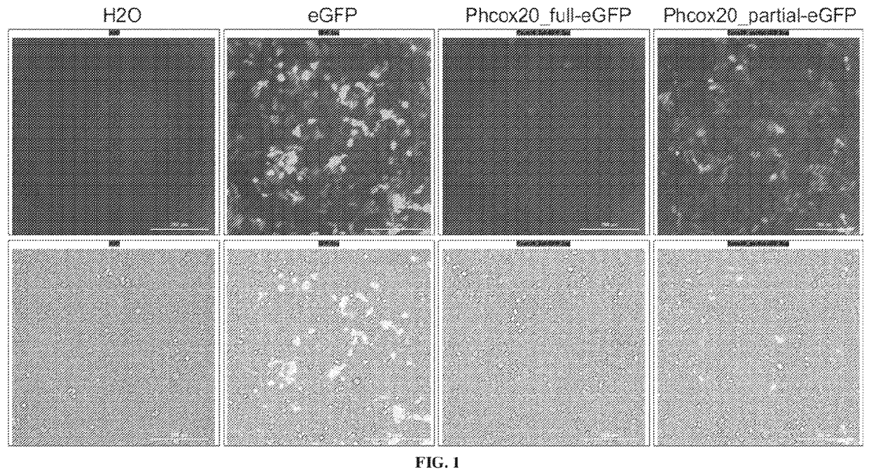

rAAV vectors were produced using a pAAV backbone and were designed to encode eGFP, COX20-201, or COX20-203 operably linked to either the fall-length or partial hCOX20 promoter. HEK293 cells were transfected with the vectors encoding eGFP operably linked to the full-length (Phcox20_full-eGFP) or partial (Phcox20_partial-cGFP) hCOX20 promoters. HEK293 cells treated with water (H2O) or transfected with a vector encoding eGFP operably linked to a constitutive promoter (cGFP) were used as control Expression of eGFP was assessed using fluorescent microscopy (FIG. 1). As expected, the cells transfected with eGFP under the control of the constitutive promoter showed the highest expression of eGFP. Notably, the cells transfected with the Phcox20_partial-eGFP showed higher eGFP expression compared to cells transfected with Phcox20_full-eGFP, indicating that the partial hCOX20 promoter drives expression more robustly than the full-length hCOX20 promoter.

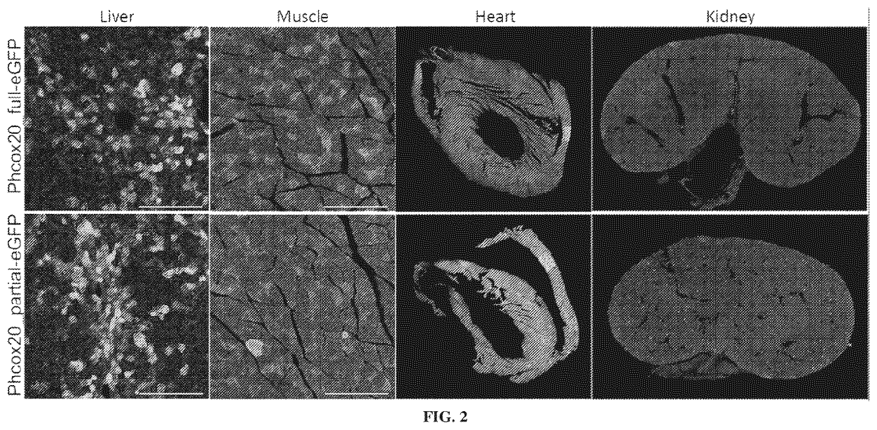

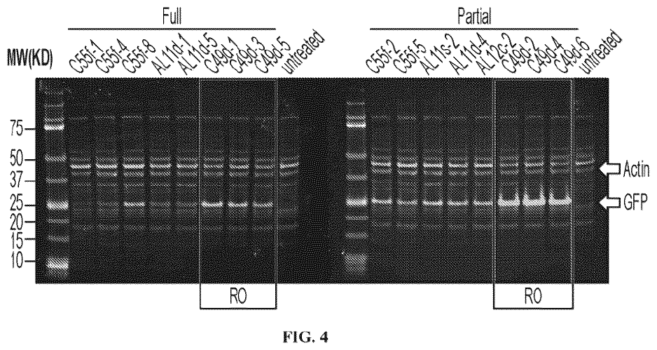

The rAAV vectors described above were then packaged in an AAV capsid protein and the resulting rAAV titers assessed (Table 1). Mice at postnatal day 9 (P9) were retro-orbitally injected with 1.5×10¹² genome copies/pup of either pAAVss.hCOX20_full_prom-eGFP or pAAVss.hCOX20_partial_prom-eGFP Expression of eGFP was assessed via immunofluorescence in the liver, muscle, heart, kidney, bruin, and spinal cord of the mice (FIGS. 2 and 4). Similar to the HEK293 in vitro expression data, the partial hCOX20 promoter drove expression of eGFP more robustly compared to the full-length hCOX20 promoter in vivo as well.

TABLE 1

| | SEQ | Titer | Volume | Retro-orbital |
|---|---|---|---|---|
| Name | ID NO: | (GC/mL) | (mL) | Injection (P9) |
| pAAVss.hCOX20_full_prom-eGFP | — | 1.69 × 10¹³ | 3.2 | 1.5 × 10¹²/pup |
| pAAVss.hCOX20_full_prom-optCox20-201 | 13 | 1.97 × 10¹³ | 2.6 | |
| pAAVss.hCOX20_full_prom-optCox20-203 | 14 | 1.35 × 10¹³ | 3 | |
| pAAVss.hCOX20_partial_prom-eGFP | — | 2.63 × 10¹³ | 3 | 1.5 × 10¹²/pup |
| pAAVss.hCOX20_partial_prom-optCox20-201 | 15 | 1.65 × 10¹³ | 3.1 | |
| pAAVss.hCOX20_partial_prom-optCox20-203 | 16 | 2.30 × 10¹³ | 2.7 | |

A second cohort of mice was administered pAAVss.hCOX20_full_prom-eGFP or PAAVss.hCOX20_partial_prom-eGFP at postnatal day 32 (P32) using intravenous injection (facial vein injection or retro-orbital injection). Livers of the mice were collected and processed to test protein expression of eGFP via biochemical analysis. Western Blot analysis of liver lysates demonstrated less eGFP protein expression in the mice of injected with pAAVss.hCOX20_full_prom-eGFP rAAVs compared to mice injected with pAAVss.hCOX20_partial_prom-cGFP rAAVs (FIG. 4, see band at ˜27 kDa). Untreated wild-type mice were used as control. Protein levels were normalized to Actin (42 kDa).

These data demonstrate that the COX20 promoter (full-length or partial, as provided in SEQ ID NOs: 9 and 10 respectively) is capable of driving expression of a transgene in mouse subjects following a systemic administration (e.g., intravenous administration).

### Example 4. Delivery of AAV Vectors Encoding COX20 Protein In Vivo

To test the ability of rAAV vectors encoding human COX20 (hCOX20) to deliver COX20 protein in vivo (e.g., to treat COX20 deficiency in subjects), (AAV vectors were generated to express the human COX20-201 or COX20-203 isoform of the COX20 gene under the control of the chicken beta-actin (CB) promoter (rAAV9.CB-hCOX20-201 or rAAV9.CB-hCOX20-203, respectively). The vectors were intravenously delivered to neonatal wild-type mice, and the subcellular localization of the resulting protein was assessed in the brain, liver, and heart by immunofluorescent co-localization analysis, hCOX20-201 and hCOX20-203 co-localized with mitochondrial markers in all tissues, demonstrating proper subcellular localization of the proteins with exogenous expression.

The mice were further monitored for changes in body weight and blood markers to assess the safety of rAAV-based gene therapy for COX20 deficiency. Although wild-type mice administered rAAV.CB-hCOX20 showed a significant decrease in body weight, no pathologic findings were observed when assessing complete blood count (CBC) and basic metabolic panel (BMP) blood tests, as well as through analysis of tissue H&E staining performed 6 months following the injections. These results indicated that the rAAV.CB-hCOX20-201 and rAAV9.CB-hCOX20-203 vectors were well-tolerated and induced a minimal toxic response in the mice.

Protein levels of mouse COX20 were measured in the mice to determine whether the administration of rAAV.CB-hCOX20-201 or rAAV9 CB-hCOX20-203 led to changes in endogenous mouse COX20 protein. Western Blot analysis demonstrated that the level of endogenous mouse COX20 remained low following administration of rAAV.CB-hCOX20-201 and rAAV9.CB-hCOX20-203. In contrast, human COX20 protein exhibited high levels of expression in tissues (including the liver, muscle, and heart) from mice injected with rAAV.CB-hCOX20-201 and rAAV9.CB-hCOX20-203, as well as in HEK293 cells transfected with rAAV.CB-hCOX20-201 and rAAV9.CB-hCOX20-203. The increase in hCOX20 levels may contribute to increased energy consumption in the mice injected with rAAV.CB-hCOX20-201 and rAAV9.CB-hCOX20-203, potentially leading to the weight loss observed in these mice.

These data demonstrate that the COX20-201 and COX20-203 isoforms (encoded by SEQ ID NOS 1 and 2, respectively) are capable of in vivo expression in mammalian subjects following intravenous administration of AAV vectors encoding such proteins.

### Example 5. Development of rAAV Vectors for Native Expression of hCOX20

In order to avoid any potential risks of unintended side effects resulting from excess COX20 protein expression, or of COX20 protein expression in unintended tissues, second-generation rAAV9-hCOX20 vectors were designed to express hCOX20-201 or hCOX20-203 under the control of the native human COX20 promoter (rAAV9.pcox20-hCOX20-201 and rAAV9,pcox20-hCOX20-201, respectively). First, the efficacy of the human COX20 promoter (pcox20) to drive expression of hCOX20 was tested in vitro, HEK293 cells were transfected with rAAV9.pcox20-hCOX20-201 and rAAV9.pcox20-hCOX20-203, and the expression of hCOX20 protein was assessed via Western Blot analysis. Human COX20 levels were increased in transfected HEK293 cells compared to untransfected cells, demonstrating the ability of the COX20 promoter to drive expression of human COX20 protein in vitro.

Next, to assess the efficacy and toxicity of the vectors in vivo, rAAV9.pcox20-hCOX20-201 or rAAV9.pcox20-hCOX20-203 was administered to wild-type mice. Western blot analysis of tissues from these mice showed enhanced levels of human COX20 protein compared to control mice (that were not administrated an rAAV encoding hCOX20 isoform). Further, the toxicity of in vivo rAAV9, pcox20-hCOX20 administration was assessed by CBC and CMP blood tests, and histological analysis. None of these assays indicated pathologic findings, indicating low levels of toxicity and suggesting the promise of an rAAV-based gene therapy in the treatment of COX20 deficiency.

### Example 6. Development of a COX20 Deficient Mouse Model

There is currently no mouse model available for COX20 deficiency. To overcome this gap, a COX20-deficient mouse model was generated to mimic the human compound heterozygous variant (COX20-Het), along with a mouse model exhibiting conditional COX20 knockout (COX20-CKO).

The mice are fully characterized, including lactate levels, weight, CMP, CBC, histological analysis, and COX20 expression and tissue distribution. To further assess the efficacy of rAAV-based therapy using these animal models, COX20-Het and COX20-CKO mice are administered rAAV vectors encoding hCOX20-201 or hCOX20-203 under the control of the full-length or partial hCOX20 promoter (rAAV9.full_pcox20-hCOX20-201, rAAV9.full_pcox20-hCOX20-203, rAAV9.partial_pcox20-hCOX20-201, or rAAV9.partial_pcox20-hCOX20-206). The mice are then fully characterized following administration of the rAAV vectors, including analysis of lactate levels, weight, CMP, CBC, histological analysis, and COX20 expression and tissue distribution. Furthermore, mitochondrial function is assessed ex vivo in cells (e.g., neuronal cells) derived from the mice. This is done, for example, by measuring oxygen consumption rate (OCR) and extracellular acidification rate (ECAR) as a readout of proper mitochondrial respiratory chain function. The mice are further monitored for pathologic neurological symptoms, such as sensory neuropathy, ataxia (e.g., early onset ataxia), dysarthria, hypotonia, dystonia, and/or ophthalmoplegia. Decreases in pathologic parameters indicate beneficial therapeutic effects in the COX20-Het and COX20-CKO mouse models of COX20 deficiency.

SELECTED SEQUENCES

>COX20-201 nucleic acid sequence (SEQ ID NO: 1)
atggctgctcctccagaacccggcgagcctgaggaacggaaggccagctgcaccagcctgcacctgagctactggaagtccctgaagct
gctgggctttctggatgtggaaaacaccccttgcgcccggcacagcatcctgtatggcagcctgggaagcgtggtggccggcttcggcca
tttcctgttcacaagcagaattagaaggtcctgcgacgtgggcgtgggaggattcatcctggtcacactgggctgctggttccactgtagata
caactacgccaagcagagaatccaggagcggatcgccagagaggaaatcaagaaaaaaatcctgtacgagggcacccacctcgaccc
cgagagaaagcacaacggctcttctagcaattga
>COX20-203 nucleic acid sequence (SEQ ID NO: 2)
atggccgctccccccgagccaggcgagcctgaggaaagaaagtccctgaagctgctgggcttcctggacgtggaaaacaccccttgcg
ccagacacagcatcctgtatggcagcctgggatctgtggtcgccggcttcggccacttcctgttcaccagcagaatccggagaagctgcg
acgtgggcgtgggaggctttatcctggtgacactgggatgttggttccactgcagatacaactacgccaagcagcggattcaggagagaat
cgcccgcgaggaaatcaagaaaaagatcctctacgagggcacccacctggatcctgaacggaaacataatggctctagcagcaactga
>COX20-201 amino acid sequence (SBQ ID NO: 3)
MAAPPEPGEPEERKASCTSLHLSYWKSLKLLGFLDVENTPCARHSILYGSLGSVVAGFG
HfLFTSRIRRSCDVGVGGFILVTLGCWFHCRYNYAKQRIQERIAREEIKKKILYEGTHLD
PERKHNGSSSN
>COX20-203 amino acid sequence (SEQ ID NO: 4)
MAAPPEPGEPEERKSLKLLGFLDVENTPCARHSILYGSLGSVVAGFGHFLFTSRIRRSCD
VGVGGFILVTLGCWFHCRYNYAKQRIQERIAREEIKKKILYEGTHLDPERKHNGSSSN
>COX20-201 rAAV nucleic acid sequence (SEQ ID NO: 5)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtcgacattgattattgactagttattaatagtaat
caattacggggtcattagttcatagcccatatatggagttccgcgttacataacttacggtaaatggcccgcctggctgaccgcccaacgacc
cccgcccattgacgtcaataatgacgtatgttcccatagtaacgccaatagggactttccattgacgtcaatgggtggagtatttacggtaaa
ctgcccacttggcagtacatcaagtgtatcatatgccaagtacgcccctattgacgtcaatgacggtaatggcccgcctggcattatgccca
gtacatgaccttatgggactttcctacttggcagtacatctacgtattagtcatcgctattaccatgtcgaggccacgttctgcttcactctc
cccatctcccccccctccccacccccaattttgtatttatttattttttaattattttgtgcagcgatgggggcggggggggggggcgcgcgc
caggcggggcggggcggggcgaggggcggggcggggcgaggcggagaggtgcggcggcagccaatcagagcggcgcgctccgaaagt
ttccttttatggcgaggcggcggcggcggcggccctataaaaagcgaagcgcgcggcgggcgggagcaagctctagcctcgagaattc
gccaccatggctgctcctccagaacccggcgagcctgaggaacggaaggccagctgcaccagcctgcacctgagctactggaagtccc
tgaagctgctgggctttctggatgtggaaaacaccccttgcgccggcacagcatcctgtatggcagcctgggaagcgtggtggccggctt
cggccatttcctgttcacaagcagaattagaaggtcctgcgacgtgggcgtgggaggattcatcctggtcacactgggctgctggttccact
gtagatacaactacgccaagcagagaatccaggagcggatcgccagagaggaaatcaagaaaaaaatcctgtacgagggcacccacct
cgaccccgagagaaagcacaacggctcttctagcaattgaggtacctctagagtcgacccgggcggcctcgaggacggggtgaactac
gcctgaggatccgatctttttccctctgccaaaaattatggggacatcatgaagccccttgagcatctgacttctggctaataaaggaaattt
attttcattgcaatagtgtgttggaattttttgtgtctctcactcggcctaggtagataagtagcatggcgggttaatcattaactacaagga
acccctagtgatggagttggccactccctctctgcgcgctcgctcgctcactgaggccgggcgaccaaggtcgcccgacgcccgggctttgcc
cgggcggcctcagtgagcgagcgagcgcgcag
>COX20-203 rAAV nucleic acid sequence (SEQ ID NO: 6)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtcgacattgattattgactagttattaatagtaa
tcaattacggggtcattagttcatagcccatatatggagttccgcgttacataacttacggtaaatggcccgcctggctgaccgcccaacgac
ccccgcccattgacgtcaataatgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaaatggcccgcctggcattatgcc
cagtacatgaccttatgggactttcctacttggcagtacatctacgtattagtcatcgctattaccatgtcgaggccacgttctgcttcactc
tccccatctcccccccctccccacccccaattttgtatttatttattttttaattattttgtgcagcgatgggggcggggggggggggcgcgc
gccaggcggggcggggcggggcgaggggcggggcggggcgaggcggagaggtgcggcggcagccaatcagagcggcgcgctccgaaagt
ttccttttatggcgaggcggcggcggcggcggccctataaaaagcgaagcgcgcggcgggcgggagcaagctctagcctcgagaattc
gccaccatggccgctccccccgagccaggcgagcctgaggaaagaaagtccctgaagctgctgggcttcctggacgtggaaaacaccc
cttgcgccagacacagcatcctgtatggcagcctgggatctgtggtcgccggcttcggccacttcctgttcaccagcagaatcaggagaag
ctgcacgtgggcgtgggaggctttatcctggtgacactgggatgttggttccactgcagatacaactacgccaagcagcggattaggag
agaatcgcccgcgaggaaatcaagaaaaagatcctctacgagggcacccacctggatcctgaacggaaacataatggctctagcagcaa
ctgaggtacctctagagtcgacccgggcggcctcgaggacggggtgaactacgcctgaggatccgatctttttccctctgccaaaaattatg
gggacatcatgaagccccttgagcatctgacttctggctaataaaggaaatttattttcattgcaatagtgtgttggaattttttgtgtctct
cactcggcctaggtagataagtagcatggcgggttaatcattaactacaaggaacccctagtgatggagttggccactccctctgcgcgctcg
ctcgctcactgaggccgggcgaccaaaggtcgcccgacgcccgggctttgcccgggcggcctcagtgagcgagcgagcgcgcag
>COX20-201 rAAV plasmid nucleic acid sequence (SEQ ID NO: 7)
ctgcgcgctcgctcgctcactgaggccgcccgggcaaagcccgggcgtcgggcgacctttggtcgcccggcctcagtgagcgagcga
gcgcgcagagagggagtgtagccatgctctaggaagatcaattcggtacaattcacgcgtcgacattgattattgactagttattaatagtaa
tcaattacggggtcattagttcatagcccatatatggagttccgcgttacataacttacggtaaatggcccgcctggctgaccgcccaacgac
ccccgcccattgacgtcaataatgacgtatgttcccatagtaacgccaatagggactttccattgacgtcaatgggtggagtatttacggtaa
actgcccacttggcagtacatcaagtgtatcatatgccaagtacgccccctattgacgtcaatgacggtaaatggcccgcctggcattatgcc
cagtacatgaccttatgggactttcctacttggcagtacatctacgtattagtcatcgctattaccatgtcgaggccacgttctgcttcactc
tccccatctcccccccctccccacccccaattttgtatttatttattttttaattattttgtgcagcgatgggggcggggggggggggcgcgc
gccaggcggggcggggcggggcgaggggcggggcggggcgaggcggagaggtgcggcggcagccaatcagagcggcgcgctccgaaagt
ttccttttatggcgaggcggcggcggcggcggccctataaaaagcgaagcgcgcggcgggcgggagcaagctctagcctcgagaattc
gccaccatggctgctcctccagaacccggcgagcctgaggaacggaaggccagctgcaccagcctgcacctgagctactggaagtccc
tgaagctgctgggctttctggatgtggaaaacaccccttgcgcccggcacagcatcctgatggcagcctgggaagcgtggtggccggctt
cggccatttcctgttcacaagcagaattagaaggtcctgcgacgtgggcgtgggaggattcatcctggtcacactgggctgctgttccact
gtagatacaactacgccaagcagagaatccaggagcggatcgccagagaggaaatcaagaaaaaaatcctgtacgagggcacccacct
cgaccccgagagaaagcacaacggctcttctagcaattgaggtacctctagagtcgacccgggcggcctcgaggacggggtgaactac
gcctgaggatccgatctttttccctctgccaaaaattatggggacatcatgaagccccttgagcatctgacttctggctaataaaggaaattt
attttcattgcaatagtgtgttggaattttttgtgtctctctcggcctaggtagataagtagcatggcgggttaatcattaactacaaggaac
ccctagtgatggagttggccactccctctctgcgcgctcgctcgctcactgaggccgggcgaccaaaggtcgcccgacgcccgggctttgccc
gggcggcctcagtgagcgagcgagcgcgcagccttaattaacctaattcactggccgtcgttttacaacgtcgtgactgggaaaaccctgg
cgttacccaacttaatcgccttgcagcacatccccctttcgccagctggcgtaatagcgaagaggcccgcaccgatcgcccttcccaacag
ttgcgcagcctgaatggcgaatgggacgcgccctgtagcggcgcattaagcgcggcgggtgtggtggttacgcgcagcgtgaccgctac
acttgccagcgccctagcgcccgctcctttcgctttcttcccttcctttctcgccacgttcgccggctttccccgtcaagctctaatcgggggc
tccctttagggttccgatttagtgctttacggcacctcgaccccaaaaaacttgattagggtgatggttcacgtagtgggccatcgccctgata
gacggtttttcgccctttgacgttggagtccacgttctttaatagtggactcttgttccaaactggaacaacactcaacccttctcggtctatt
cttttgatttataagggattttgccgatttcggcctattggttaaaaatgagctgatttaacaaaaatttaacgcgaattttaacaaaatatta
acgcttacaatttaggtggcacttttcggggaaatgtgcgcggaacccctatttgtttatttttctaaatacattcaaatatgtatccgctcat
gagacaataaccctgataaatgcttcaataatattgaaaaggaagagtatgagtattcaacatttccgtgtcgcccttattcccttttttgcgg
cattttgccttcctgtttttgctcacccagaaacgctggtgaaagtaaaagatgctgagatcagttgggtgcacgagtgggttacatcgaactg
gatctcaacagcggtaagatccttgagagttttcgccccgaagaacgttttccaatgatgagcacttttaaagttctgctatgtggcgcggtat
tatcccgtattgacgccgggcaagagcaactcggtcgccgcatacactattctcagaatgacttggttgagtactcaccagtcacagaaaagca
tcttacggatggcatgacagtaagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccggagctgaatgaagccataccaa
acgacgagcgtgacaccacgatgcctgtagcaatggcaacaacgttgcgcaaactattaactggcgaactacttactctagcttcccggcaa
caattaatagactggatggaggcggataaagttgcaggaccacttctgcgctcggcccttccggctggctggtttattgctgataaatctgga
gccggtgagcgtgggtctcgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcatata
tactttagattgatttaaaacttcatttttaatttaaaaggatctaggtgaagatcctttttgataatctcatgaccaaaatcccttaacgtga
gttttcgttccactgagcgtcagaccccgtagaaaagatcaaaggatcttcttgagatcctttttttctgcgcgtaatctgctgcttgcaaaca
aaaaaaccaccgctaccagcggtggtttgtttgccggatcaagagctaccaactctttttccgaaggtaactggcttcagcagagcgcagatac
caaatactgttcttctagtgtagccgtagttaggccaccacttcaagaactctgtagcaccgcctacatacctcgctctgctaatcctgttacc
agtggctgctgccagtggcgataagtcgtgtcttaccgggttggactcaagacgatagttaccggataaggcgcagcggtcgggctgaacgggg
ggttcgtgcacacagcccagcttggagcgaacgacctacaccgaactgagatacctacagcgtgagctatgagaaagcgccacgcttcc
cgaagggagaaaggcggacaggtatccggtaagcggcagggtcggaacaggagagcgcacgagggagcttccagggggaaacgcc
tggtatctttatagtcctgtcgggtttcgccacctctgacttgagcgtcgatttttgtgatgctcgtcaggggggcggagcctatggaaaaacg
ccagcaacgcggcctttttacggttcctggccttttgctggccttttgctcacatgttctttcctgcgttatcccctgattctgtggataaccg
tattaccgcctttgagtgagctgataccgctcgccgcagccgaacgaccgagcgcagcgagtcagtgagcgaggaagcggaagagcgccca
atacgcaaaccgcctctccccgcgcgttggccgattcattaatgcagctggcacgacaggtttcccgactggaaagcgggcagtgagcgc
aacgcaattaatgtgagttagctcactcattaggcaccccaggctttacactttatgcttccggctcgtatgttgtgtggaattgtgagcggat
aacaatttcacacaggaaacagctatgaccatgattacgccagatttaattaaggccttaattagg
>COX20-203 rAAV plasmid nucleic acid sequence (SEQ ID NO: 8)
ctgcgcgctcgctcgctcactgaggccgcccgggcaaagcccgggcgtcgggcgacctttggtcgcccggctcagtgagcgagcga
gcgcgcagagagggagtgtagccatgctctaggaagatcaattcggtacaattcacgcgtcgacattgattattgactagttattaatagtaat
caattacggggtcattagttcatagcccatatatggagttccgcgttacataacttacggtaaatggcccgcctggctgaccgcccaacgacc
cccgcccattgacgtcaataatgacgtatgttcccatagtaacgccaatagggactttccattgacgtcaatgggtggagtatttacggtaaac
tgcccacttggcagtacatcaagtgtatcatatgccaagtacgccccctattgacgtcaatgacggtaaatggcccgcctggcattatgccca
gtacatgaccttatgggactttcctacttggcagtacatctacgtattagtcatcgctattaccatgtcgaggccacgttctgcttcactctcc
ccatctcccccccctccccacccccaattttgtatttatttattttttaattattttgtgcagcgatgggggcggggggggggggcgcgcgcca
ggcggggcggggcggggcgaggggcggggcggggcgaggcggagaggtgcggcggcagccaatcagagcggcgcgctccgaaagt
ttccttttatggcgaggcggcggcggcggcggccctataaaaagcgaagcgcgcggcgggcgggagcaagctctagcctcgagaattc
gccaccatggccgctccccccgagccaggcgagcctgaggaaagaaagtccctgaagctgctgggcttcctggacgtggaaaacaccc
cttgcgccagacacagcatcctgtatggcagcctgggatctgtggtcgccggcttcggccacttcctgttcaccagcagaatccggagaag
ctgcgacgtgggcgtgggaggctttatcctggtgacactgggatgttggttccactgcagatacaactacgccaagcagcggattcaggag
agaatcgcccgcgaggaaatcaagaaaaagatcctctacgagggcacccacctggatcctgaacggaaacataatggctctagcagcaa
ctgaggtacctctagagtcgacccgggcggcctcgaggacggggtgaactacgcctgaggatccgatctttttccctctgccaaaaattatg
gggacatcatgaagccccttgagcatcgacttctggctaataaaggaaatttattttcattgcaatagtgtgttggaattttttgtgtctctca
ctcggcctaggtagataagtagcatggcgggttaatcattaactacaaggaacccctagtgatggagttggccactccctctctgcgcgctcgc
tcgctcactgaggccgggcgaccaaaggtcgcccgacgcccgggctttgcccgggcggcctcagtgagcgagcgagcgcgcagcctta
attaacctaattcactggccgtcgttttacaacgtcgtgactgggaaaaccctggcgttacccaacttaatcgccttgcagcacatcccccttt
cgccagctggcgtaatagcgaagaggcccgcaccgatcgcccttcccaacagttgcgcagcctgaatggcgaatgggacgcgccctgta
gcggcgcattaagcgcggcgggtgtggtggttacgcgcagcgtgaccgctacacttgccagcgccctagcgcccgctcctttcgctttctt
cccttcctttctcgccacgttcgccggctttccccgtcagctctaaatcgggggctccctttagggttccgatttagtgctttacggcacctcg
accccaaaaaacttgattagggtgatggttcacgtagtgggccatcgccctgatagacggtttttcgccctttgacgttggagtccacgttctt
aatagtggactcttgttccaaactggaacaacactcaaccctatctcggtctattcttttgatttataagggattttgccgatttcggcctatt
ggttaaaaaatgagctgatttaacaaaaatttaacgcgaattttaacaaaatattaacgcttacaatttaggtggcacttttcggggaaatgtg
cgcggaacccctatttgtttatttttctaaatacattcaaatatgtatccgctcatgagacaataaccctgataaatgcttcaataatattgaa
aaggaagagtatgagtattcaacatttccgtgtcgcccttattcccttttttgcggcattttgccttcctgtttttgctcacccagaaacgctg
gtgaaagtaaaagatgctgaagatcagttgggtgcacgagtgggttacatcgaactggatctcaacagcggtaagatccttgagagttttcgcc
ccgaagaacgttttccaatgatgagcacttttaaagttctgctatgtggcgcggtattatcccgtattgacgccgggcaagagcaactcggtcg
ccgcatacactattctcagaatgacttggttgagtactcaccagtcacagaaaagcatcttacggatggcatgacagtaagagaattatgcagt
gctgccataaccatgagtgataacactgcggccaacttacttctgacaacgatcggaggaccgaaggagctaaccgcttttttgcacaacatgg
gggatcatgtaactcgccttgatcgttgggaaccggagctgaatgaagccataccaaacgacgagcgtgacaccacgatgcctgtagcatggca
acaacgttgcgcaaactattaactggcgaactacttactctagcttcccggcaacaattaatagactggatggaggcggataaagttgcagg
accacttctgcgctcggcccttccggctggctggtttattgctgataaatctggagccggtgagcgtgggtctcgcggtatcattgcagcact
ggggccagatggtaagccctcccgtatcgtagttatctacacgacggggagtcaggcaactatggatgaacgaaatagacagatcgctga
gataggtgcctcactgattaagcattggtaactgtcagaccaagtttactcatatatactttagattgatttaaaacttcatttttaatttaaa
aggatctaggtgaagatccttttgataatctcatgaccaaaatcccttaacgtgagtttcgttccactgagcgtcagaccccgtagaaaagatc
aaaggatcttcttgagatcctttttttctgcgcgtaatctgctgcttgcaaacaaaaaaaccaccgctaccagcggtggtttgtttgccggatc
aagagctaccaactctttttccgaaggtaactggcttcagcagagcgcagataccaaatactgttcttctagtgtagccgtagttaggccacca
cttcaagaactctgtagcaccgcctacatacctcgctctgctaatcctgttaccagtggctgctgccagtggcgataagtcgtgtcttaccggg
ttggactcaagacgatagttaccggataaggcgcagcggtcgggctgaacggggggttcgtgcacacagcccagcttggagcgaacgacct
acaccgaactgagatacctacagcgtgagctatgagaaagcgccacgcttcccgaagggagaaaggcggacaggtatccggtaagcgg
cagggtcggaacaggagagcgcacgagggagcttccagggggaaacgcctggtatctttatagtcctgtcgggtttcgccacctctgactt
gagcgtcgatttttgtgatgctcgtcaggggggcggagcctatggaaaaacgccagcaacgcggcctttttacggttcctggccttttgctg
gccttttgctcacatgttctttcctgcgttatcccctgattctgtggataaccgtattaccgcctttgagtgagctgataccgctcgccgcagc
cgaacgaccgagcgcagcgagtcagtgagcgaggaagcggaagagcgcccaatacgcaaaccgcctctccccgcgcgttggccgattca
ttaatgcagctggcacgacaggtttcccgactggaaagcgggcagtgagcgcaacgcaattaatgtgagttagctcactcattaggcaccc
caggctttacactttatgcttccggctcgtatgttgtgtggaattgtgagcggataacaatttcacacaggaaacagctatgaccatgattacg
ccagatttaattaaggccttaattagg
>Full-length Human COX Promoter (SEQ ID NO: 9)
GCCTCGTGTAATAATATTTAATTAAGAAAAAGCCCGGTCCCATAGCTCATGTCTCTA
ATCCCAGCACTTTGGGAGGGTGAGACAGGAGGATCACTTTAGTCCAGGAGTTTGAG
ACCACCCTGGGCAACAAAGTGACACACCATCCCCCACCTCCCGTCTCTACAAATAA
TTGTTTTTAAGAAGGATAAAAGAGTTTGAAAAGAGAGACATCAAACTGTTAGTTAT
ATTTACCTTAAAAAGGAGAGAAAGAACTGAGATTTTTACTTTCCTTTCTATAACTGT
TAAACAATTAAAAATGGAAATATTATATGCAATTTTGTGAAAATAAAAATTTTAAA
ATAGGCCAGGCGCAGTGGCTCACGCCTGTAATCCCAGCACTCTGGAAACCAGAGGC
AGGCGTATCACCTGAGGTCAGGAGTTTGAGGCCAGCCTGGCCAACATAGTGAAACC
CTTTCTTCTACTAAAAATACAAAAATTAGCCGGGTGTGGTGGCAGCCGCCTGTAATCC
CACAATGTCTACTCTAGCAAATTTTCAAAGTCATAATAAAAATAAAATAGGATGAC
TCGGTCGTCTTTTAGCTGGTACTTTTCCAGTTTTCCTTTTACAACATAATCAACAATG
ACTTCAAAAGGATTTCCCCCTCCTTCATGGAATATTACTACCTTCAACACGGCCTTT
CAGATCCCGAGTTCAAACCATACTTAGCATTATAATTTCAGCAGAGAATCCAGAAT
TGGCAGGATATCACCAGTAGGGTGACTTTTTTTCTTTTCCCTAGCAAACAGTAGAGC
AGTATGTAGTTTCTCCCCCCGCCCAGACGGAGTTTCCCTCTTTCTCCCAGCCTCGAG
TACAGTAAAAAACCTCAAAAAAAAAAATGTGACACATAAATACACAACCTTAACTG
TGTATTTTACTGGGATTGTAGAAAGGTATCAAATGGGCATGCAAGTAAGGTCCTTAC
GGAGTAAGAAAACGCCCAATGAACAGTACTTAATATGAATTCGACATCGTTAACAC
TGAGCGCTTGCTGTGTGCCAGCGCCCAGCATAATTCGAAGCAGCATGACTTTGCAC
AATGACATATTCTACAAGTCCAGTTTCCTAAGGAGTGCCCTTAGGTTACATATAACC
TAAACGTGAAATGTAGAAGTACATAAAGGAGCCATAAAGTTAAAGGGGAAAAATT
ATAAAAATTGCATTCTATTATTTTGATGGCTGAAAAATGGGCTCGACCCTGCATGCA
CGCGTTCTCCATTGAGCTCGACAATATGCAGTATTTTGTTTCACGTTATGTCGCAGTC
TTAGGGAGGACTGAAACGTGGAACACGACTTCACATCAAAAATGCACGTTGCAGAG
GCATTTTTCCTCTTAAAGCCACTTAGTAATGAAACTATAAAAGGGAGGAAGTGGGG
TGGGCGTGGGAGCCCCGTTTGCGCAAGGCAGGCGCGGGGGACTAAATGGCTGCTGG
TGCAGAGTGGGGCTGCCCCGGCACCAAGGGCTCGTCCCGTGCCCCGAAGGACAAA
CGACACTTAGGGGACGAGCGCCGCCGAGCCAGCGCGGAGCGCCTTCCGGCTCGGT
GGGCTGGGGGGGGCTGGGGGCTGGGGCCGGGCCGACCCCCGCGGTGGGGCCCACC
CCGCAGGACAAAGCAGCGGCCCCGCCCCTCGGCGCGCGCCCGAGGCTCGTGCGTGT
GAGCACGCGCGCCGGAGACGCTTAGAAAAGAAAGTGTGACCCGTTCGACGGAACA
AAGGACATGACAGCCCGGCCCCGTGCGGCCACTGCGGAGCGTTAGGAACATTCCCT
AAAATGGCGGCCGGCCCGTCGGAACAGGGGGGGGGGGGCGGCGCGTGGGGGGGG
GACCGGGAGGGGCCCGGCCAGCCGGGCTTCTGCTTCCCCGACCCCGGCCGTGCAGG
CCCGGTGGAGTCGCGGAGTAGTCCGGCGGGGCGCGCGCCGCGGGTGGGCGGTCGG
TAAGGACGTTCTCCGCGGAGCTCCTAGGCCCGGAGCACGTGTCACTGGCTTCTCTGC
CACCCATGGGCTTGAAACTATTTTAACTGTTCCTAGTCCTTATCCCAAGCCTCCAAC
ATTTTAATCTAAGTGGCCGGGACAAAATTGACCAAAACTAGGGGGAACATAGGAGT
AATTGCTGAGGGATGACTTGATTGTACTACGATGAGATGAAGGGTTAAGATTTCTTC
CCCATCACCAGTAGCCTCCCTAGCTGGTGTCCCTGTCTCCATACTCTCCTGAATCCA
CCTTCCACTATTGTCCGCTGGCCCTTTCCAGCCTCTAAACTGGACAGTTTTCATTCCT
CTGCCTAACATCCAGTCGTCCTAATATATACTTAAGATATTCTCCCAGCTTGGAAAT
TCTGCTCACTTCTTGCTCTTGCTTGCTTCTCTGGTGGGCTTCCATCCAAATTTCGCCA
TGACATCAGATCATATGCTTAATATTAACAAATATTGTATAAATTGTTTTCATACTT
AATATCCAACAACTAGACTGATCAAGACAGGAAAAATCCCTTTAAGGAGCTCACAT
TCTACTGGAGGAATCAATAAGCAAGACTTCTACTGAGCCAGTCATGCAAAGATCCA
GGAGAAAGGCAGCATGTGTTCGTTCGGTAATAACCATCTCGGCTTGTTAGAATGGC
AAGGCCACTGTACCTGGACTAGTGGACAAGAGTAGGATATGAAAGAGCATGGGAT
CATGTAGGCCCTTTGGAGCAATGCTAAGGATTCTCAGCATAATCGGAGGCTACAGA
GGACTTATTATAGTCATCTGATACACATTTTTATGAGTAGAATCAAGATCTGAAAGG
TACTC
>Partial Haman COX Promoter (SEQ ID NO: 10)
CAATGTCTACTCTAGCAAATTTTCAAAGTCATAATAAAAATAAAATAGGATGACTC
GGTGGTGTTTTAGCTGGTACTTTTCCAGTTTTCCTTTTACAACATAATCAACAATGAC
TTCAAAAGGATTTCCCCCTCCTTCATGGAATATTACTACCTTCAACACGGCCTTTCA
GATCCGGAGTTCAAAGCATAGTTAGCATTATAATTTCAGCAGAGAATCCAGAATTG
GCAGGATATCACCAGTAGGGTGACTTTTTTTCTTTTCCCTAGCAAACAGTAGAGCAG
TATGTAGTTTCTCCCCCCGCCCAGACGGAGTTTCGCTCTTTCTCCCAGGCTGGAGTA
CAGTAAAAAACCTCAAAAAAAAAAATGTGACACATAAATACACAACCTTAACTGTG
TATTTTACTGGGATTGTAGAAAGGTATCAAATGGGCATGCAAGTAAGGTCCTTACG
GAGTAAGAAAACGCCCAATGAACAGTACTTAATATGAATTCGACATCGTTAACACT
GAGCGCTTGCTGTGTGCCAGCGCGCAGCATAATTCGAAGCAGCATGACTTTGCACA
ATGACATATTCTACAAGTCCAGTTTCCTAAGGAGTGCCCTTAGGTTACATATAACCT
AAACGTGAAATGTAGAAGTACATAAAGGAGGCATAAAGTTAAAGGGGAAAAATTA
TAAAAATTGCATTGTATTATTTTGATGGCTGAAAAATGGGCTCGACCCTGCATGCAC
GCCTTCTCCATTGAGCTCGACAATATGCAGTATTTTGTTTCACGTTATGTCGCAGTCT
TAGGGAGGAGTGAAACGTGGAACACGACTTCACATCAAAAATGCACGTTGCAGAG
GCATTTTTCCTGTTAAAGCCACTTAGTAATGAAACTATAAAAGGGAGGAAGTGGGG
TGGGCGTGGGAGCCCCGTTTGCGCAAGGCAGGCGCGGGGGACTAAATGGCTGCTGG
TGCAGAGTGGGGCTGCCCCGGCACCAAGGGCTCGTCCCGTGGCCGGAAGGACAAA
GGACACTTAGGGGACGAGGGCCGCCGAGCCAGCGCGGAGCGCCTTCCGGGTCGGT
GGCCTGGGGGGGGCTGGGGGCTGGGGCCGGGCCGACCCCCGCGGTGGGGCCCACC
CCGCAGGACAAAGCAGCGGCCCCCCCCCTGGGCGCGCGCCCGAGGCTCGTGCGTGT
CAGCACGCGCGCCGGAGACGGTTAGAAAAGAAAGTGTGACCCGTTCGACGGAACA
AAGGACATGACAGCCCGGCCCCGTGCGGCCACTGCGGAGCGTTAGGAACATTCCCT
AAAATGGCGGCCGGCGCGTCGGAACAGGGGGGGGGGGGCGGCGCGTGGGGGGGG
GACGGGGAGGGGCGCGGCCAGCCGGGCTTCTGCTTGCGCGACCCCGGCGGTGCAGG
GCGGGTGGAGTCGCGGAGTAGTCCGGCGGGGCGCGCGCCGCGGGGGGCGGTGGG
TAAGGACGTTCTCCGCGGAGCTCCTAGGCCCGGAGCACGTGTCACTGGCTTCTCTGC
CACCCATGGGCTTGAAACTATTTTAACTGTTCCTAGTCCTTATCCCAAGCCTCCAAC
ATTTTAATCTAAGTGGCCGGGACAAAATTGACCAAAACTAGGGGGAACATAGGAGT
AATTGCTGAGGGATGACTTGATTGTACTACGATGAGATGAAGGGTTAAGATTTCTTC
CCCATCACCAGTAGCCTCCCTAGCTGGTGTCCCTGTCICCATACTCTCCTGAATCCA
CCTTCCACTATTGTCCGCTGGCCCTTTCCAGCCTGTAAACTGGACAGTTTTCATTGCT
CTGCCTAACATCCAGTCGT
>E1438691/prom (SEQ ID NO: 11)
TGTTAAAGCCACTTAGTAATGAAACTATAAAAGGGAGGAAGTGGGGTGGGCGTGG
CAGCCCCGTTTGCGCAAGGCAGGCGCGGGGGACTAAATGGCTGCTGGTGCAGACTG
GGGCTGCCCCGGCACCAAGGGCTCGTCCCGTGGCCGGAAGGACAAAGGACACTTAG
GGGACGAGGGCCCCCGAGCCAGCGCGGAGCGCCTTCCGGGTCGGTGGGCTGGGGC
GGGCTGGGGGCTGGGGCCGGGCCGACCCCCGCGGTGGGGCCCACCCCGCAGGACA
AAGCAGCGGCCCCGCCCCTCGGCGCGCCCCCGAGGCTCGTGCGTGTGAGCACGCGC
GCCGGAGACGGTTAGAA
>E1438692/prom (SEQ ID NO: 12)
AAGAAAGTGTGACCCGTTCGACGGAACAAAGGACATGACAGCCCGGCCCCGTGCG
GCCACTGCGGAGCGTTAGGAACATTCCCTAAAATGGCGGCCGGCGCGTCGGAACAG
GGGGGGAGGCGGCGGCGCGTGGGGGGGGGACGGGGAGGGGCGCGGCCAGCCGGG
CTTCTGCTTCCGCGACCCCGGCGGTGCAGGGCGGGTGGAGTCGCGGAGTAGTCC
EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either.” “one of,” “only one of,” or “exactly one of.” “Consisting essentially of” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second.” “third,” etc., in the claims to modify a claim element does not by itself connote any priority, precedence, or order of one claim element over another or the temporal order is which acts of a method are performed, but we used merely as labels to distinguish one claim element having a certain name from another element having a same name (but for use of the ordinal team) to distinguish the claim elements.

The terms “about” and “substantially” preceding a numerical value represent ±10% of the recited americal value.

What is claimed is:

1. An isolated nucleic acid comprising a transgene encoding a Cytochrome C Oxidase Assembly Factor COX20 (COX20) protein, wherein the transgene comprises a nucleic acid sequence having at least 70%, 80%, 85%, 90%, 95%, 98%, 99%, or 100% identity to SEQ ID NOs: 1 or 2.

2. The isolated nucleic acid of claim 1, wherein the COX20 protein comprises an amino acid sequence having at least 90%, 95%, 98%, 99%, or 100% identity to an amino acid sequence set forth in SEQ ID NOs: 3 or 4.

3. The isolated nucleic acid of claim 1 or 2, further comprising a promoter.

4. The isolated nucleic acid of claim 3, wherein the promoter is a constitutive promoter, an inducible promoter, or a tissue-specific promoter.

5. The isolated nucleic acid of claim 3 or 4, wherein the promoter is a COX20 promoter, optionally a human COX20 promoter.

6. The isolated nucleic acid of claim 5, wherein the COX20 promoter comprises a sequence having at least 70%, 75%, 80%, 85%, 90%, 95%, 98%), 99%, or 100% identity to any one of SEQ ID NOs: 9-12.

7. The isolated nucleic acid of any one of claims 1-6, further comprising at least one adeno-associated virus (AAV) inverted terminal repeat (ITR).

8. The isolated nucleic acid of claim 7, wherein the at least one AAV ITR is an AAV2 ITR.

9. The isolated nucleic acid of claim 7 or 8, wherein the at least one AAV ITR is a truncated ITR (ATTR).

10. The isolated nucleic acid of any one of claims 1-9 comprising a sequence having at least 70%, 75%, 80%, 85%, 90%, 95%, 98%, 99%, or 100% identity to any one of SEQ ID NOs: 5-8 or 13-16.

11. A recombinant adeno-associated virus (rAAV) comprising the isolated nucleic acid of any one of claims 1-10 and at least one AAV capsid protein.

12. The rAAV of claim 11, wherein the at least one AAV capsid protein is an AAV1, AAV2, AAV3, AAV4, AAV5, AAV6, AAV7, AAV8, AAV9, AAV.PHP-Eb, AAV.rh10 capsid protein, or a variant thereof.

13. A vector comprising the isolated nucleic acid of any one of claims 1-10.

14. The vector of claim 13, wherein the vector is a plasmid or a viral vector.

15. The vector of claim 14, wherein the viral vector is an adenoviral vector, an adeno-associated virus vector, a lentiviral vector, a retroviral vector, or a Baculovirus vector

16. A recombinant adeno-associated virus (rAAV) comprising:
(1) an isolated nucleic acid comprising a transgene encoding a Cytochrome C Oxidase Assembly Factor COX20 (COX20; protein, wherein the transgene comprises a nucleic acid sequence having at least 70%, 75%, 80%, 85%, 90%, 95%, 98%, 99%, or 100% identity to SEQ ID NOs: 1 or 2; and
(ii) at least one AAV capsid protein.

17. The rAAV of claim 16, wherein the COX20 protein comprises an amino acid sequence having at least 90%, 95%, 98%, 99%, or 100% identity to an amino acid sequence set forth in SEQ ID NOs: 3 or 4.

18. The rAAV of any one of claim 11, 12, 16, or 17, wherein the rAAV is a self-complementary AAV (scAAV) or a single-stranded AAV (ssAAV).

19. The rAAV of any one of claim 11, 12, or 16-18, wherein the at least one AAV capsid protein has a tropism for nervous system cells, optionally neuronal cells.

20. The rAAV of any one of claim 11, 12, or 16-19, wherein the at least one AAV capsid protein is an AAV1, AAV2, AAV3, AAV4, AAV5, AAV6, AAV7, AAV8, AAV9, AAV.PHP-Eb, AAV.th10 capsid protein, or a variant thereof.

21. A composition comprising:
(a) the isolated nucleic acid of any one of claims 1-10 or the rAAV of any one of claim 11, 12, or 16-20; and
(b) a pharmaceutically acceptable excipient.

22. A host cell comprising the isolated nucleic acid of any one of claims 1-10 or the rAAV of any one of claim 11, 12, or 16-20.

23. The host cell of claim 22, wherein the host cell is a bacterial cell, a mammalian cell, or an insect cell.

24. The host cell of claim 23, wherein the mammalian cell is a human cell.

25. A method for treating Cytochrome C Oxidase Assembly Factor COX20 (COX20) deficiency in a subject, the method comprising administering to the subject the isolated nucleic acid of any one of claims 1-10, the rAAV of any one of claim 11, 12, or 16-20, or the composition of claim 21.

26. A method of decreasing a lactate level in a subject, the method comprising administering to the subject the isolated nucleic acid of any one of claims 1-10, the rAAV of any one of claim 11, 12, or 16-20, or the composition of claim 21.

27. A method for preventing or treating Cytochrome C Oxidase Assembly Factor COX20 (COX20) deficiency in a subject, the method comprising administering to the subject an isolated nucleic acid comprising a transgene encoding a COX20 protein, wherein the transgene comprises a nucleic acid sequence having at least 70%, 75%, 80%, 85%, 90%, 95%, 98%, 99%, or 100% identity to SEQ ID NOs: 1 or 2.

28. A method of decreasing a lactate level in a subject, the method comprising administering to the subject an isolated nucleic acid comprising a transgene encoding a Cytochrome C Oxidase Assembly Factor COX20 (COX20) protein, wherein the transgene comprises a nucleic acid sequence having at least 70%, 75%, 80%, 85%, 90%, 95%, 98%, 99%, or 100% identity to SEQ ID NOs: 1 or 2.

29. The method of claim 27 or 28, wherein the COX20 protein comprises an amino acid sequence having at least 70%, 75%, 80%, 85%, 90%, 95%, 98%, 99%, or 100% identity to an amino acid sequence set forth in SEQ ID NOs: 3 or 4.

30. The method of any one of claims 27-29, wherein the isolated nucleic acid further comprises a promoter.

31. The method of claim 30, wherein the promoter is a constitutive promoter, an inducible promoter, or a tissue-specific promoter.

32. The method of claim 30 or 31, wherein the promoter is a COX20 promoter, optionally a human COX20 promoter.

33. The method of claim 32, wherein the COX20 promoter comprises a sequence having at least 70%, 75%, 80%, 85%, 90%, 95%, 98%, 99%, or 100% identity to any one of SEQ ID NOS: 9-12.

34. The method of any one of claims 27-33, wherein the isolated nucleic acid further comprises at least one adeno-associated virus (AAV) invented terminal repeat (ITR), optionally wherein the at least one AAV ITR is an AAV2 ITR or a truncated ITR (ΔITR).

35. The method of any one of claims 25-34, wherein the subject is a human and/or the subject has at least one mutation in a COX20 gene.

36. The method of any one of claims 25-35, wherein the administering is performed using a systemic injection, an injection directly into the central nervous system of the subject, or an intravenous administration.

37. The method of any one of claims 25-36, wherein the administration results in a decrease in a lactate level in the subject relative to the lactate level in the subject prior to the administration.
